SEQUENCE LISTING (27 sequences)

SEQ ID NO: 1 - DNA, 18 bp, synthetic construct
  misc_feature 1..18: note = Synthetic primer
  source 1..18: mol_type = other DNA
cgcgggtaaagctaccaa

SEQ ID NO: 2 - DNA, 25 bp, synthetic construct
  misc_feature 1..25: note = Synthetic primer
  source 1..25: mol_type = other DNA
ctaagtaaacttgtttcccacagtc

SEQ ID NO: 3 - DNA, 23 bp, synthetic construct
  misc_feature 1..23: note = Synthetic primer
  source 1..23: mol_type = other DNA
gctctcaacagatcttctcactc

SEQ ID NO: 4 - DNA, 22 bp, synthetic construct
  misc_feature 1..22: note = Synthetic primer
  source 1..22: mol_type = other DNA
tctggtttgagggaagctaaag

SEQ ID NO: 5 - DNA, 20 bp, synthetic construct
  misc_feature 1..20: note = Synthetic primer
  source 1..20: mol_type = other DNA
ctgtcgcgtgtctctcatta

SEQ ID NO: 6 - DNA, 21 bp, synthetic construct
  misc_feature 1..21: note = Synthetic primer
  source 1..21: mol_type = other DNA
tccactcgagagtagacagaa

SEQ ID NO: 7 - DNA, 21 bp, synthetic construct
  misc_feature 1..21: note = Synthetic primer
  source 1..21: mol_type = other DNA
ctaaccgaaacccgtctcttc

SEQ ID NO: 8 - DNA, 22 bp, synthetic construct
  misc_feature 1..22: note = Synthetic primer
  source 1..22: mol_type = other DNA
gtttccctgcaccttctctatc

SEQ ID NO: 9 - DNA, 35 bp, synthetic construct
  misc_feature 1..35: note = Synthetic primer
  source 1..35: mol_type = other DNA
cgcggtcgactctccaagtgatacactccatatcc

SEQ ID NO: 10 - DNA, 35 bp, synthetic construct
  misc_feature 1..35: note = Synthetic primer
  source 1..35: mol_type = other DNA
cgcggtcgacgagatgggtcgggttttgcgggaat

SEQ ID NO: 11 - DNA, 35 bp, synthetic construct
  misc_feature 1..35: note = Synthetic primer
  source 1..35: mol_type = other DNA
cgcggtcgacatggaacgaaacgagaaaacaagtc

SEQ ID NO: 12 - DNA, 35 bp, synthetic construct
  misc_feature 1..35: note = Synthetic primer
  source 1..35: mol_type = other DNA
cgcgggatccgagatgggtcgggttttgcgggaat

SEQ ID NO: 13 - DNA, 20 bp, synthetic construct
  misc_feature 1..20: note = Synthetic primer
  source 1..20: mol_type = other DNA
gacgcttgttgctgctgttg

SEQ ID NO: 14 - DNA, 27 bp, synthetic construct
  misc_feature 1..27: note = Synthetic primer
  source 1..27: mol_type = other DNA
gcttcctattatatcttcccaaattac

SEQ ID NO: 15 - DNA, 21 bp, synthetic construct
  misc_feature 1..21: note = Synthetic primer
  source 1..21: mol_type = other DNA
tgggaattcgtcatgccactc

SEQ ID NO: 16 - DNA, 20 bp, synthetic construct
  misc_feature 1..20: note = Synthetic primer
  source 1..20: mol_type = other DNA
gacgcttgttgctgctgttg

SEQ ID NO: 17 - DNA, 19 bp, synthetic construct
  misc_feature 1..19: note = Synthetic primer
  source 1..19: mol_type = other DNA
gacgaggctgagctacaaa

SEQ ID NO: 18 - DNA, 23 bp, synthetic construct
  misc_feature 1..23: note = Synthetic primer
  source 1..23: mol_type = other DNA
tggttcacgtagtgggccatccg

SEQ ID NO: 19 - DNA, 19 bp, synthetic construct
  misc_feature 1..19: note = Synthetic primer
  source 1..19: mol_type = other DNA
gacgaggctgagctacaaa

SEQ ID NO: 20 - DNA, 25 bp, synthetic construct
  misc_feature 1..25: note = Synthetic primer
  source 1..25: mol_type = other DNA
agggcgtagtaataattaaatgctg

SEQ ID NO: 21 - protein, 811 aa, Arabidopsis thaliana
MKLSTSGLGQQGHEGEKCLNSELWHACAGPLVSLPSSGSRVVYFPQGHSEQVAATTNKEVDGHIPNYPSLPPQLICQLHNVTMHADVETDEVYAQMTLQPLTPEEQKETFVPIELGIPSKQPSNYFCKTLTASDTSTHGGFSVPRRAAEKVFPPLDYTLQPPAQELIARDLHDVEWKFRHIFRGQPKRHLLTTGWSVFVSAKRLVAGDSVIFIRNEKNQLFLGIRHATRPQTIVPSSVLSSDSMHIGLLAAAAHASATNSCFTVFFHPRASQSEFVIQLSKYIKAVFHTRISVGMRFRMLFETEESSVRRYMGTITGISDLDSVRWPNSHWRSVKVGWDESTAGERQPRVSLWEIEPLTTFPMYPSLFPLRLKRPWHAGTSSLPDGRGDLGSGLTWLRGGGGEQQGLLPLNYPSVGLFPWMQQRLDLSQMGTDNNQQYQAMLAAGLQNIGGGDPLRQQFVQLQEPHHQYLQQSASHNSDLMLQQQQQQQASRHLMHAQTQIMSENLPQQNMRQEVSNQPAGQQQQLQQPDQNAYLNAFKMQNGHLQQWQQQSEMPSPSFMKSDFTDSSNKFATTASPASGDGNLLNFSITGQSVLPEQLTTEGWSPKASNTFSEPLSLPQAYPGKSLALEPGNPQNPSLFGVDPDSGLFLPSTVPRFASSSGDAEASPMSLTDSGFQNSLYSCMQDTTHELLHGAGQINSSNQTKNFVKVYKSGSVGRSLDISRFSSYHELREELGKMFAIEGLLEDPLRSGWQLVFVDKENDILLLGDDPWESFVNNVWYIKILSPEDVHQMGDHGEGSGGLFPQNPTHL

SEQ ID NO: 22 - protein, 831 aa, Oryza glaberrima
MSSSSAASIGPPQPPPPPAPPEEGGWLEKKCLNSELWHACAGPLVCLPTVGTRVVYFPQGHSEQVRLAYVKVAASTNKEVEGHIPNYPNLPAQLICQLHDVTMHADVETDEVYAQMTLQPLNPQEQNDAYLPAEMGIMSKQPTNYFCKTLTASDTSTHGGFSVPRRAAERVFPPLDFTQQPPAQELIARDIHDIEWKFRHIFRGQPKRHLLTTGWSVFVSAKRLVAGDSVLFIWNEKNQLLLGIRRASRPQTVMPSSVLSSDSMHIGLLAAAAHAAATNSRFTIFYNPRASPSEFVIPLSKYIKAVFHTRISVGMRFRMLFETEESSVRRYMGTITEVSDADPVRWPSSYWRSVKVGWDESTAGERPPRVSLWEIEPLTTFPMYPSLFPLRVKHPWYSGVASLHDDSNALMWLRGVAGEGGFQSLNFQSPGIGSWGQQRLHPSLLSSDHDQYQAVVAAAAASHSGGYLKQQFLHLQQPMQSPQEHCNLNPLLQQQILQQASQQQIINPDAQNIQTMLSPSAIQQQLQQLQQMQQVQNDQKQKIQPDQSYQVPTSAVLPSPTSLPSHLREKFGFSDPNANSSSFITSSSSDNMLDSSFLQGSSKAVDLSRFNQPVASEQQQQQQQQQAWKQKFMGSQSVSFGGSVLHNSPTSKDGSVENKIGRDVQNQSLFSPQVDSSSLLYNMVPNLTSNVSDGNLSTIPSGSTYLQNAMYGCLDDSSGLLQNTGENDPATRTFVKVYKSGSVGRSLDITRFSNYAELREELGQMFGIKGQLDDPDRSGWQLVFVDRENDVLLLGDDPWESFVNSVWYIKILSPEDVHKMGKQGNDPRYLS

SEQ ID NO: 23 - protein, 908 aa, Oryza sativa
MKLSPSAGGVSDQPPSPPEVAEEQKCLNSELWHACAGPLVSLPAVGSRVVYFPQGHSEQVAASTNKEMESQIPNYPNLPPQLICQLHNVTMHADAETDEVYAQMTLQPLSPQELKDPFLPAELGTASKQPTNYFCKTLTASDTSTHGGFSVPRRAAEKVFPPLDFTQQPPAQELMAKDLHGNEWKFRHIFRGQPKRHLLTTGWSVFVSAKRLVAGDSVLFIWNDSNQLLLGIRRANRPQTVMPSSVLSSDSMHIGLLAAAAHAASTNSRFTIFYNPRASPSEFVIPLAKYVKAVYHTRISVGMRFRMLFETEESSVRRYMGTITGISDLDPVRWMNSHWRSVKVGWDESTAGERQPRVSLWEIEPLTTFPMYPSPFPLRLKRPWPTGLPSLYGGKEDDLASSLMWLRDSQNTGFQSLNFGGLGMSPWMQPRLDSSLLGLQPDMYQTIAAAAALQNTTKQVSPAMLQFQQPQNIVGRSSLLSSQILQQAQPQFQQMYHQNINGNSIQGHSQPEYLQQPLQHCQSFNEQKPQLQPQQQQQESHQQQPQHQQMQQQKHLSNFQTVPNALSVFSQLSSTPQSTPSTLQTVSPFSQQHNFPDTNISCLSPSNVSSMHDTLRSFPSEAASDLPGVPRITPVPVSDPWSSKRVAVESTITSRPHDISSQIENFDLTPSSIPQNSTLAPLPGRECLVDQDGSSDPQNHFLFGVNIDSQSLLMQDGIPSLHNENSSSTIPYSTSNFLSPSQDDYPLSQTLTTPGCLDESGYVPCSDNADQVKRPHATFVKVYKSGTVGRLLDITRFSSYHELRSEVGRLFGLEGQLEDPLRSGWQLVFVDREDDVLLVGDDPWQEFVNSVSCIKILSPQEVQQMGKPGIELFSTSARRLGNSCDNYMSRQESRSLSTGIASVGSVEF

SEQ ID NO: 24 - protein, 818 aa, Oryza sativa
MSSSSAASIGPPQPPPPPAPPEEEKKCLNSELWHACAGPLVCLPTVGTRVVYFPQGHSEQVAASTNKEVEGHIPNYPNLPAQLICQLHDVTMHADVETDEVYAQMTLQPLNPQEQNDAYLPAEMGIMSKQPTNYFCKTLTASDTSTHGGFSVPRRAAERVFPPLDFTQQPPAQELIARDIHDIEWKFRHIFRGQPKRHLLTTGWSVFVSAKRLVAGDSVLFIWNEKNQLLLGIRRASRPQTVMPSSVLSSDSMHIGLLAAAAHAAATNSRFTIFYNPRASPSEFVIPLSKYIKAVFHTRISVGMRFRMLFETEESSVRRYMGTITEVSDADPVRWPSSYWRSVKVGWDESTAGERPPRVSLWEIEPLTTFPMYPSLFPLRVKHPWYSGVASLHDDSNALMWLRGVAGEGGFQSLNFQSPGIGSWGQQRLHPSLLSSDHDQYQAVVAAAAASQSGGYLKQQFLHLQQPMQSPQEHCNLNPLLQQQILQQASQQQIINPDAQNIQTMLSPSAIQQQLQQLQQMQQVQNDQKQKIQPDQSYQVPTSAVLPSPTSLPSHLREKFGFSDPNANSSSFITSSSSDNMLDSSFLQGSSKAVDLSRFNQPVASEQQQQQQQAWKQKFMGSQSVSFGGSVLHNSPTSKDGSVENKIGRDVQNQSLFSPQVDSSSLLYNMVPNLTSNVSDGNLSTIPSGSTYLQNAMYGCLDDSSGLLQNTGENDPATRTFVKVYKSGSVGRSLDITRFSNYAELREELGQMFGIKGQLDDPDRSGWQLVFVDRENDVLLLGDDPWESFVNSVWYIKILSPEDVHKMGKQGNDPRYLS

SEQ ID NO: 25 - protein, 908 aa, Oryza sativa; identical to SEQ ID NO: 23

SEQ ID NO: 26 - protein, 853 aa, Oryza sativa
MATAEVGGGGGEGDAAAAAVARAGGGGGGGGGGGEDALFTELWSACAGPLVTVPRVGEKVFYFPQGHIEQVEASTNQVGEQRMQLYNLPWKILCEVMNVELKAEPDTDEVYAQLTLLPELKQQEDNGSTEEEVPSAPAAGHVRPRVHSFCKTLTASDTSTHGGFSVLRRHADECLPPLDMSRQPPTQELVAKDLHGVEWRFRHIFRGQPRRHLLQSGWSVFVSAKRLVAGDAFIFLRGENGELRVGVRRAMRQQTNVPSSVISSHSMHLGVLATAWHAVNTGTMFTVYYKPRTSPAEFVVPYDRYMESLKRNYSIGMRFKMRFEGEEAPEQRFTGTIVGMGDSDPAGWPESKWRSLKVRWDEASSIPRPERVSPWQIEPAVSPPPVNPLPVPRTKRLRPNATALPADSSAIAKEAATKVVVESEPNGTQRTFQTQENATPKSGFGNSSELESAQKSIMRPSGFDREKNNTPIQWKLGSDGWMQMSKPESYSEMLSGFQPPKDVQTPQGFCSLPEQITAGHSNFWHTVNAQYQDQQSNHNMFPSSWSFMPPNTRLGLNKQNYSMIQEAGVLSQRPGNTKFGNGVYAALPGRGTEQYSGGWFGHMMPNSHMDDTQPRLIKPKPLVVAHGDVQKAKGASCKLFGIHLDSPAKSEPLKSPSSVVYDGTPQTPGATEWRRPDVTEVEKCSDPSKAMKPLDTPQPDSVPEKPSSQQASRNMSCKSQGVSTRSCKKVHKQGIALGRSVDLTKFNGYEELIAELDDMFDFNGELKGPKKEWMVVYTDNEGDMMLVGDDPWIEFCDMVHKIFIYTREEVQRMNPGTLNSRSEDSHANSMERGSVGREMRGCLSTSSLNSENC

SEQ ID NO: 27 - protein, 848 aa, Zea mays
MSSSSAASIGQQPPEEEKKCLNSELWHACAGPLVCLPTVATRVVYFPQGHSEQVAASTNKEVDGHIPNYPNLPPQLICQLHDVTMHADVETDEVYAQMTLQPLNPQEQNDPYLPAEMGIMSKQPTNYFCKTLTASDTSTHGGFSVPRRAAERVFPPLVWYSVVCRISHSSLQHRSLLHGIFMMSSGSSGISSEASQPKRHLLTTGWSVFVSAKRLVAGDSVLFIWNEKNQLLLGIRRASRPQTVMPSSVLSSDSMHIGLLAAAAHAAATNSRFTIFFNPRASPSEFVIPLSKYIKAVFHTRISVGMRFRMLFETEESSVRRYMGTITEVSDADPVRWPSSYWRSVKVGWDESTAGERPPRVSLWEIEPLTTFPMYPSLFPLRVKHPWYSGVAALHGTLQILMLVAITLSAAPLVLWLFTCVISVTATNNALMWLRGVAGEGGFQSLNFQSPGVGSWGQQRLHPSLLSNDHDQYQAVVAAAAASQSDGYLKQQFLHLQQPMQSPQEQCNLNPLLQQQILQQGSQQQMVSPDAQNIQSVLNPNAIQQQLQQFQQMQHAHNDQKQKIQPDQPYQVPSSAVLSSPTSLPSHLREKFGFSDPNVNSSSFISSSSNENMLESNFLQGSSKSVDLSRFNQPVVSEQQQQQAWKQKFICSQSMSFGGSVSLNSPTTKDGPVDNKIGRDVQNQTLFSPQVDSSSLLYNMVPNLTSNVADNNISTIPSGSTYLQSPMYGCLDDSSGLLQNTGENDPTTRTFVKVYKSGSVGRSLDITRFSNYAELREELGQMFGIKGQLDDPDRSGWQLVFVDRENDVLLLGDDPWESFVNSVWYIKILSPEDVHKMGKPGNDPRYLS

(19) United States
(12) Patent Application Publication
(10) Pub. No.: US 2023/0309481 A1
(43) Pub. Date: Oct. 5, 2023

(54) MODIFICATION OF PLANT GENE EXPRESSION TO EFFECT INCREASED SEED SIZE AND YIELD

(71) Applicant: Saint Louis University, St. Louis, MO (US)

(72) Inventors: Wenyan XIAO, St. Louis, MO (US); Arthur BARTELS, St. Louis, MO (US); Qiang HAN, St. Louis, MO (US)

(73) Assignee: Saint Louis University, St. Louis, MO (US)

(21) Appl. No.: 18/162,399

(22) Filed: Jan. 31, 2023

Related U.S. Application Data
(60) Provisional application No. 63/325,481, filed on Mar. 30, 2022.

(51) Int. Cl. A01H 1/00; A01H 1/04

(52) U.S. Cl. CPC A01H 1/121; A01H 1/04

(57) ABSTRACT

The present disclosure is directed to modified plant cells and plants having increased seed number and seed size. Studies identified genes such as ARF8, YDK1, RNA Pol IV, RNA Pol V, DCL3, AGO6, CMT3 and DRM2 as linked to seed characteristics and the inventors therefore propose modulating the expression and/or activity of these target genes in plants as an approach to increase/improve seed production.

### PRIORITY CLAIM

This application claims benefit of priority to U.S. Provisional Application Ser. No. 63/325,481, filed Mar. 30, 2022, the entire contents of which are hereby incorporated by reference.

### REFERENCE TO A SEQUENCE LISTING

This application contains a Sequence Listing XML, which has been submitted electronically and is hereby incorporated by reference in its entirety. Said Sequence Listing XML, created on Apr. 28, 2023, is named USTLP0143US_Sequence_Listing.xml and is 38 kB in size.

### BACKGROUND

### 1. Field of the Disclosure

The present disclosure relates generally to the fields of botany, molecular biology and plant genetics and breeding. More particular, the disclosure relates to modification of gene expression and gene function in plants to produce larger seeds and/or greater number of seeds per plant.

### 2. Background

Seed represents the major nutrient source for humans and domesticated animal. Little is known about the genetic mechanism that contributes to the overall seed size even though its importance is known. Seed is the product of a double fertilization event in angiosperms that occurs in the female gametophyte of the ovule. During fertilization, one sperm cell fertilizes the egg cell to generate a diploid embryo, which gives rise to the new developing plant. The second sperm fertilizes the diploid central cell to generate a triploid endosperm, which supports embryo and seedling growth (Brown, Lemmon et al., 1999; Drews and Yadegari, 2002; Gehring, Choi et al., 2004; Drews, Wang et al., 2011). The last major component of the seed is the seed coat, which is maternally derived tissues that encloses the embryo sac. Embryo and endosperm coordinate their development with the surrounding seed coat. This complex interplay between the endosperm, embryo, and seed coat and the parent plant controls the overall development of the seed.

The plant hormone auxin was first isolated in 1926 and has been shown to affect many aspects of plant development from cell division, elongation, differentiation, embryonic development, root and stem tropisms, apical dominance, and flower formation. Most of these diverse processes are regulated through auxin-regulated gene expression. Two types of transcription factor families are required for controlling expression of auxin response genes. One of these, the auxin response factor (ARF) family, functions by binding to auxin response elements (AuxREs) on promoters of auxin response genes, activating or repressing the auxin response genes, and recruiting a second family of transcription factors, the Aux/IAA repressors, that confer an auxin response to the genes.

Certain mutations and experiments have shown that knocking out certain transcription factors can cause large effects on seed size. Large seeds in Arabidopsis can be generated by mutation of the APETALA2 (AP2) transcription factor (Jofuku, Omidyar et al., 2005; Ohto, Fischer et al., 2005) and the size of many organs, including seeds, is increased by ectopic expression of the AINTEGUMENTA (ANT) transcription factor (Mizukami and Fischer 2000). Even AUXIN RESPONSE FACTOR 2 (ARF2), a member of a family of transcription factors that mediate gene expression in response to auxin was shown to increase seed size and weight (Schruff, Spielman et al., 2006).

### SUMMARY

Thus, in accordance with the present disclosure, there is provided a modified plant cell comprising gene with a mutation as compared to a comparable plant cell that results in the increase or decrease in the expression and/or activity of a corresponding gene product as compared to said comparable plant cell selected from the group consisting of ARF8, YDK1, RNA Pol IV, RNA Pol V, DCL3, AGO6, CMT3 and DRM2. The plant may be Arabidopsis thaliana, corn, soybean, canola, rice, wheat, oat or barley. The plant cell may be homozygous for said mutation or heterozygous for said mutation. More than mutation may be present in said plant cell and/or more than one corresponding gene product may have modified expression or activity in said plant cell. The mutation may be (a) located in said gene result in an amino acid substitution or truncation of said corresponding gene product; (b) located in a regulatory sequence that controls expression of said corresponding gene product; or (c) a heterologous nucleic acid encoding an agonist or antagonist of expression and/or activity of said corresponding gene product.

Also provided is a living plant comprising a modified plant cell as described herein. Also provided is a seed obtained from propagating such a plant, a plant obtained by breeding two plants, one or both of which is/are such a plant, or progeny, a descendant, part or propagule of such a plant

In another embodiment, there is provided a method of propagating a plant comprising (a) planting a seed comprising a gene with mutation as compared to a comparable plant cell that results in the increase or decrease in the expression or activity of a corresponding gene product as compared to said comparable plant cell, wherein the gene is selected from the group consisting of ARF8, YDK1, RNA Pol IV, RNA Pol V, DCL3, AGO6, CMT3 and DRM2; and (b) cultivating a plant grown from said seed.

In yet another embodiment, there is provided a method of increasing or decreasing in the expression and/or activity of a gene product in a plant cell selected from the group consisting of ARF8, YDK1, RNA Pol IV, RNA Pol V, DCL3, AGO6, CMT3 and DRM2 comprising contacting said plant cell with an agonist or antagonist of expression and/or activity of said gene product. The plant cell may be located in a living, intact plant. The agonist or antagonist may be an organochemcial compound a nucleic acid, such as an mRNA or an expression vector, or a protein or peptide.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description.

It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Effects of the arf8-2 mutation on seed size in Arabidopsis. WT, Wild-type Col-0 self-pollinated seeds; arf8-2, the homozygous arf8-2 mutant self-pollinated seeds.

FIG. 2. The arf8-2 mutation affects seed development. Nomarski photographs of wild-type (WT) and the homozygous arf8-2 mutant embryos at 1 to 6 DAP are shown.

FIG. 3. Effects of the ydk1 mutation on seed size in Arabidopsis. WT, Wild-type Col-0; ydk1, the homozygous ydk1.

FIG. 4. Expression of ANT, CYCD3, and AP2 in wild-type Col-0, arf8-2 and ydk1 mutants. RNA was isolated from seeds of wild-type and the mutants at 3 days after pollination (DAP) and 7 DAP and used for RT-PCR examination of expression of ANT, CYCD3, and AP2. ACT expression was used as a loading control.

FIG. 5. ARF8 alignment with Oryza glaberrima.

FIG. 6. ARF8 alignment with Oryza sativa Japonica.

FIG. 7. ARF8 alignment with Oryza sativa Indica.

FIG. 8. ARF8 multiple organism sequence alignments.

FIG. 9. Effects of the nrpd1-3 and nrpe1-11 mutation on seed size in Arabidopsis. WT, Wild-type Col-0; nrpd1-3, homozygous nrpd1-3; nrpe1-11, homozygous nrpe1-11.

FIG. 10. Effects of the dcl3-1, ago6-2, drm2-2 and cmt3-11 mutation on seed size in Arabidopsis. WT, Wild-type Col-0; dcl3-1, homozygous dcl3-1; ago6-2, homozygous ago6-2; drm2-2, homozygous drm2-2; cmt3-11, homozygous cmt3-11.

FIG. 11. Maternal and paternal effects of the nrpd-1-3 mutation on seed size in Arabidopsis. For each cross, the genotype of the maternal parent is indicated first, followed by the paternal parent. WT, Wild-type parent; nrpd1-3, homozygous nrpd1-3 parent.

FIG. 12. Maternal and paternal effects of the nrpe-1-11 mutation on seed size in Arabidopsis. For each cross, the genotype of the maternal parent is indicated first, followed by the paternal parent. WT, Wild-type parent; nrpe1-11, homozygous nrpe1-11 parent.

FIG. 13. ARF8 gene expression in the arf8-2 homozygous mutant, WT Col-0, and arf8-2 homozygous mutant complemented with the ARF8 transgene.

FIG. 14. Maternal and paternal effects of the arf8-2 mutation on seed size in Arabidopsis. For each cross, the genotype of the maternal parent is indicated first, followed by the paternal parent. WT, Wild-type parent; arf8-2, the homozygous arf8-2 mutant.

FIGS. 15a-b. Embryo and Endosperm nuclei of wild-type and the arf8-2 mutant collected through Fluorescence-activated cell sorting. DAPI (4′,6-diamidino-2-phenylindole) staining is shown and the gates are set to collect embryo and endosperm cells. (FIG. 15a) 2n and 4n nuclei associated with embryo cells are shown by the peaks. On the left is the WT embryo cells and on the right is the arf8-2 mutant cells. (FIG. 15b) 3n and 6n nuclei associated with endosperm cells are shown by the peaks. On the left is the WT embryo cells and on the right is the arf8-2 mutant cells.

FIG. 16. Maternal and Paternal Effects of the ydk1 mutation on seed size in Arabidopsis. For each cross, the genotype of the maternal parent is indicated first, followed by the paternal parent. WT, Wild-type parent; ydk1, homozygous ydk1 parent.

### DESCRIPTION OF ILLUSTRATIVE EMBODIMENTS

As discussed above, seed represents the main nutrient source for humans and domesticated animals and is the product of a double fertilization event in angiosperms. As such, seed size is an essential trait for increasing yield and/or nutritional value. Unfortunately, while seed size is an important component of crop yield, it is usually negatively correlated with seed number per silique or plant. For example, ARF2 mutations have been found that can result in larger seed size, but they also cause seed number per silique to decrease, thus making it impractical to increase total yield for crop plants. Mutations in in other genes that would result in larger seeds, but without the significant negative effect—decrease in seed number per silique—would be highly valued. However, at present, the ideal combination of increased seed size and unchanged or increase seed number per plant is unmet goal for many plants.

The inventors have discovered an Arabidopsis gene called AUXIN RESPONSE FACTOR 8 (ARF8) in which null mutations result in larger seeds. The mutant arf8-2 seed develops normally but seeds were approximately 24% larger than wild-type (WT). Larger seed in the arf8-2 mutant is due to increased cell numbers in both embryo and endosperm—arf8-2 had 154% and 46% more cells in embryo and endosperm than WT at 9 days after pollination (DAP), respectively. They also found seed number per silique was not significantly reduced and the weight of the decreased seed number does not account for the increased seed weight from larger seed. At the molecular level, the increased cell numbers of embryo and endosperm in arf8-2 could be due to increase activity of three transcription factors involved in cell division during seed development: AINTEGUMENTA (ANT), APETALA2 (AP2), and CYCD3 (a D-type cyclin). YADOKARI 1 (YDK1), an auxin responsive GRETCHEN HAGEN 3 (GH3) gene functions downstream of ARF8. Furthermore, mutations in YDK1 also cause increased seed size. The data presented shows a possible regulatory network between auxin, auxin transcription factors, and cell divisions to cause larger seed size. They have also identified homologous genes for ARF8, AP2, CYCD3, and YDK1 in corn, oat, rice, soybean, barley, canola, and wheat. By knocking out or knocking down of these genes or altering auxin signaling pathway, seed size and yield in these crop plants should also be increased.

These and other aspects of the disclosure are described in detail below.

### I. PLANT SPECIES

A. Arabidopsis

Arabidopsis (rockcress) is a genus in the family Brassicaceae. They are small flowering plants related to cabbage and mustard. This genus is of great interest since it contains thale cress (Arabidopsis thaliana), one of the model organisms used for studying plant biology and the first plant to have its entire genome sequenced. Changes in thale cress are easily observed, making it a very useful model.

Arabidopsis thaliana, the thale cress, mouse-ear cress or Arabidopsis, is a small flowering plant native to Eurasia and Africa. A. thaliana is considered a weed; it is found along the shoulders of roads and in disturbed land.

A winter annual with a relatively short lifecycle, A. thaliana is a popular model organism in plant biology and genetics. For a complex multicellular eukaryote, A. thaliana has a relatively small genome around 135 megabase pairs. It was the first plant to have its genome sequenced and is a popular tool for understanding the molecular biology of many plant traits, including flower development and light sensing.

Arabidopsis thaliana is an annual (rarely biennial) plant, usually growing to 20-25 cm tall. The leaves form a rosette at the base of the plant, with a few leaves also on the flowering stem. The basal leaves are green to slightly purplish in color, 1.5-5 cm long, and 2-10 mm broad, with an entire to coarsely serrated margin; the stem leaves are smaller and unstalked, usually with an entire margin. Leaves are covered with small, unicellular hairs called trichomes. The flowers are 3 mm in diameter, arranged in a corymb; their structure is that of the typical Brassicaceae. The fruit is a siliqua 5-20 mm long, containing 20-30 seeds. Roots are simple in structure, with a single primary root that grows vertically downward, later producing smaller lateral roots. These roots form interactions with rhizosphere bacteria such as Bacillus megaterium.

A. thaliana can complete its entire lifecycle in six weeks. The central stem that produces flowers grows after about 3 weeks, and the flowers naturally self-pollinate. In the lab, A. thaliana may be grown in Petri plates, pots, or hydroponics, under fluorescent lights or in a greenhouse.

Thousands of natural inbred accessions of A. thaliana have been collected from throughout its natural and introduced range. These accessions exhibit considerable genetic and phenotypic variation, which can be used to study the adaptation of this species to different environments. Botanists and biologists began to research A. thaliana in the early 1900s, and the first systematic description of mutants was done around 1945. A. thaliana is now widely used for studying plant sciences, including genetics, evolution, population genetics, and plant development. Although A. thaliana has little direct significance for agriculture, several of its traits make it a useful model for understanding the genetic, cellular, and molecular biology of flowering plants.

In the 1980s, A. thaliana started to become widely used in plant research laboratories around the world. It was one of several candidates that included maize, petunia, and tobacco. The latter two were attractive, since they were easily transformable with the then-current technologies, while maize was a well-established genetic model for plant biology. The breakthrough year for A. thaliana as a model plant was 1986, in which T-DNA-mediated transformation and the first cloned A. thaliana gene were described.

Due to the small size of its genome, and because it is diploid, Arabidopsis thaliana is useful for genetic mapping and sequencing, with about 157 megabase pairs and five chromosomes, A. thaliana has one of the smallest genomes among plants. It was long thought to have the smallest genome of all flowering plants, but that title is now considered to belong to plants in the genus Genlisea, order Lamiales, with Genlisea tuberosa, a carnivorous plant, showing a genome size of approximately 61 Mbp. It was the first plant genome to be sequenced, completed in 2000 by the Arabidopsis Genome Initiative. The most up-to-date version of the A. thaliana genome is maintained by the Arabidopsis Information Resource.

The genome encodes ˜27,600 protein-coding genes and about 6,500 non-coding genes. However, the Uniprot database lists 39,342 proteins in their Arabidopsis reference proteome. Among the 27,600 protein-coding genes 25,402 (91.8%) are now annotated with “meaningful” product names, although a large fraction of these proteins is likely only poorly understood and only known in general terms (e.g., as “DNA-binding protein without known specificity”). Uniprot lists more than 3,000 proteins as “uncharacterized” as part of the reference proteome.

The plastome of A. thaliana is a 154,478 base-pair-long DNA molecule, a size typically encountered in most flowering plants. It comprises 136 genes coding for small subunit ribosomal proteins (rps, in yellow: see figure), large subunit ribosomal proteins (rpl, orange), hypothetical chloroplast open reading frame proteins (ycf, lemon), proteins involved in photosynthetic reactions (green) or in other functions (red), ribosomal RNAs (rrn, blue), and transfer RNAs (trn, black).

The mitochondrial genome of A. thaliana is 367,808 base pairs long and contains 57 genes. There are many repeated regions in the Arabidopsis mitochondrial genome. The largest repeats recombine regularly and isomerize the genome. Like most plant mitochondrial genomes, the Arabidopsis mitochondrial genome exists as a complex arrangement of overlapping branched and linear molecules in vivo.

Genetic transformation of A. thaliana is routine, using Agrobacterium tumefaciens to transfer DNA into the plant genome. The current protocol, termed “floral dip,” involves simply dipping flowers into a solution containing Agrobacterium carrying a plasmid of interest and a detergent. This method avoids the need for tissue culture or plant regeneration.

The A. thaliana gene knockout collections are a unique resource for plant biology made possible by the availability of high-throughput transformation and funding for genomics resources. The site of T-DNA insertions has been determined for over 300,000 independent transgenic lines, with the information and seeds accessible through online T-DNA databases. Through these collections, insertional mutants are available for most genes in A. thaliana.

Characterized accessions and mutant lines of A. thaliana serve as experimental material in laboratory studies. The most commonly used background lines are Ler (Landsberg erecta), and Col, or Columbia. Other background lines less-often cited in the scientific literature are Ws, or Wassilewskija, C24, Cvi, or Cape Verde Islands, Nossen, etc.

Trichome formation is initiated by the GLABROUS1 protein. Knockouts of the corresponding gene lead to glabrous plants. This phenotype has already been used in gene editing experiments and might be of interest as visual marker for plant research to improve gene editing methods such as CRISPR/Cas9.

In 2005, scientists proposed that A. thaliana possessed an alternative to previously known mechanisms of DNA repair, producing an unusual pattern of inheritance, but the phenomenon observed (reversion of mutant copies of the HOTHEAD gene to a wild-type state) was later suggested to be an artifact because the mutants show increased outcrossing due to organ fusion.

B. Other Species

The present disclosure and methods described therein can also be applied to a variety of other plant species including, but not limited to, Soybean (Glycine max), Canola (Brassica napa), Maize (Zea mays), Rice (Orya sativa indica), Rice (Orya sativa japonica), Rice (Orya glamberrima), Wheat (T. aestivum), Wheat (T. durum), Oats (Avena sativa), and Barley (Hordeum vulgare).

### II. TARGET GENES

The discussion below relates to genes according to their primary Arabidopsis designation but the relevance of these genes is not limited to Arabidopsis but rather to corresponding homologues in other plant species.

A. ARF8

Auxin response factors (ARFs) are transcriptional factors that bind specifically to the DNA sequence 5′-TGTCTC-3′ found in the auxin-responsive promoter elements (AuxREs). Arabidopsis ARF8 seems to act as a transcriptional activator and form heterodimers with Aux/IAA proteins that may alter their ability to modulate early auxin response genes expression. Auxin response factor 8 (ARF8) can regulate both stamen and gynoecium maturation and promotes jasmonic acid production. It is partially redundant with ARF6. ARF8 is involved in fruit initiation and acts as an inhibitor to stop further carpel development in the absence of fertilization and the generation of signals required to initiate fruit and seed development. Auxin response factor 8 protein and mRNA accession numbers are AT5G37020 (TAIR Accession: 1009132777; UniProKB; Q9FGV1-1; GenPept: 15239481) and AT5G37020.1 (TAIR Accession: 2152644 and 1009064563; Gen Bank Accession: 2152644 and NM_123060), respectively.

Homologs of Arabidopsis ARF8 have been identified in soybean, canola, maize, rice, wheat, oat and barley:

| Plant Species | Gene Locus |
|---|---|
| Soybean (Glycine max) | Glyma18g05330.1, |
| | Phy00290u4_SOYBN |
| Soybean (Glycine max) | I1LM27, Glyma11g31940.1, |
| | Phy0028PYY_SOYBN |
| Soybean (Glycine max) | I1MBP7, Glyma14g38940.1, |
| | Phy0028VJX_SOYBN |
| Soybean (Glycine max) | I1JHQ8, Glyma02g40650.1, |
| | Phy002895F_SOYBN |
| Canola (Brassica napa) | LOC106445029 |
| Canola (Brassica napa) | LOC106435561 |
| Canola (Brassica napa) | LOC111199287 |
| Canola (Brassica napa) | LOC106426279 |
| Maize (Zea mays) | K7U7V1, |
| | GRMZM2G475882_P01, |
| | Phy003UEZO_MAIZE |
| Maize (Zea mays) | AOA096R641, |
| | GRMZM2G078274_P01, |
| | Phy002GYAE_MAIZE |
| Rice (Orya sativa indica) | B8ARE1, |
| | Phy003VK11_ORYSI, |
| | BGIOSGA014119-PA |
| Rice (Orya sativa japonica) | LOC_OS04g57610.1, |
| | Phy003SEMV_OrySJ |
| Rice (Orya glamberrima) | ORGLA02G0045800 |
| Rice (Orya glamberrima) | ORGLA04G0254900 |
| Wheat (T. aestivum) | TraesCS5D02G045700 |
| Wheat (T. aestivum) | TraesCS5A02G038300 |
| Wheat (T. aestivum) | TraesCS5B02G039800 |
| Wheat (T. durum) | TRITD5Bv1G01090 |
| Wheat (T. durum) | TRITD5Av1G016860 |
| Wheat (T. durum) | TRITD2Bv1G251950 |
| Wheat (T. durum) | TRITD2Av1G284350 |
| Oats (Avena sativa) | CTG23271 |
| Oats (Avena sativa) | CTG58239 |
| Oats (Avena sativa) | CTG17224 |
| Oats (Avena sativa) | CTG21042 |
| Barley (Hordeum vulgare) | HORVU2Hr1G121110 |
| Barley (Hordeum vulgare) | HORVU5Hr1G009650 |
| Barley (Hordeum vulgare) | HORVU7Hr1G106280 |
| Barley (Hordeum vulgare) | HORVU0Hr1G011310 |

B. YDK1

YDK1 is a member of the GH3 gene family. In Arabidopsis, there are 20 GH3 gene homologs and YDK1 belongs to group II. Most of the members of this group adenylate IAA in vitro. It is known that regulation of IAA conjugate formation is an important component in the maintenance of IAA levels. Thus, some of the group II GH3 proteins including YDK1 might function as negative components in auxin signaling by controlling the level of the active form of auxin. Its expression is regulated by auxin and light. It is expressed mainly in roots and flowers and overexpression of YDK1 causes de-etiolated phenotype. YDK1 protein and mRNA accession numbers are AT4G37390 (TAIR Accession: 1009126051; UniProLKB: Q9SZT9-2; GenPept: 15235538) and AT4G37390.1 (TAIR Accession: 2126364 and 4515116463; GenBank Accession: NM_119902), respectively.

C. RNA Pol IV

RNA polymerase IV (RNAP IV or Pol IV) is an enzyme that synthesizes small interfering RNA (siRNA) in plants, which silence gene expression. RNAP IV belongs to a family of enzymes that catalyze the process of transcription known as RNA Polymerases, which synthesize RNA from DNA templates. Discovered via phylogenetic studies of land plants, genes of RNAP IV are thought to have resulted from multistep evolution processes that occurred in RNA Polymerase II phylogenies. Such an evolutionary pathway is supported by the fact that RNAP IV is composed of 12 protein subunits that are either similar or identical to RNA polymerase II and is specific to plant genomes. Via its synthesis of siRNA, RNAP IV is involved in regulation of heterochromatin formation in a process known as RNA directed DNA Methylation (RdDM). RNA Pol IV protein and mRNA accession numbers are AT1G63020 (TAIR Accession: 1009106685; UniProtKB: Q9LQ02-1; GenPept: 30696792) and AT1G6302.1 (TAIR Accession: 1005036814 and 4010733243; Gen-Bank Accession: NM_104980), respectively.

Phylogenetic studies of land plants have led to the discovery of RNA Polymerase IV. Analysis of the largest (RPD1) and second-largest subunits (RPD2) of RNAP IV were analogous to the Blast searches of RNAP II genes. Genes for RPD1 and RPD2 were found in all terrestrial plants, and the largest gene was found in the algal taxon, Charale. Further analysis of the origin of the protein indicates a gene duplication event of the largest subunit which suggested that the duplication event occurred after the divergence of Charales and land plants and algae. Specifically, the largest subunit in RNAP II formed RPD1 through a duplication event and the RPD2 gene arose due to a divergence. Evidence of these duplication events imply that the RNAP IV genes come from RNAP II phylogenies in a multistep process. In other words, the divergence of the first subunit is the first step of multiple in the evolution of new RNAPs. RNAP IV also shares multiple subunits with RNAP II, in addition to the largest and second largest subunits, which was also suggested by continuous duplication events of particular lineages.

Arabidopsis expresses two forms of RNAP IV, formerly referred to as RNAP IVa and RNAP IVb, which differ at the largest subunit and have non-redundant actions. Efficient silencing of transposons requires both RNAP IV forms while only RNAP IVa is required for basal silencing. This finding suggested the requirement of both forms for the mechanism of transposon methylation. Later experiments have shown that what was once thought to be two forms of RNAP IV are actually two structurally and functionally distinct polymerases. RNAP IVa was specified to be RNAP IV while RNAP IVb became known as RNAP V.

RNA Polymerase IV is composed of 12 protein subunits that are either similar or identical to the 12 subunits composing RNA Polymerase II. Only four subunits distinguish RNAP IV structure from RNAP II and RNAP V. RNA Polymerase V differs from RNAP II by six subunits, indicating that both RNAP IV and RNAP V evolved from RNAP II in plants. In Arabidopsis, two unique genes were found to encode subunits that distinguish RNAP IV from RNAP II. The largest subunit is encoded by NRPD1 (formerly NRPD1a), while the second largest subunit is encoded by NRPD2 and is shared with RNAP V. These subunits contain carboxyl-terminal domains (CTDs) which are necessary for the production of 20-30% of the siRNAs produced by RNA Polymerase IV yet are not required for DNA methylation.

There is evidence that RNA Polymerase IV (RNAP IV) is responsible for producing heterochromatin, as dysfunction of either RNAP IV catalytic subunit (NRPD1 and NRPD2) disrupts the formation of heterochromatin. As heterochromatin is the silenced portion of DNA, it is formed when RNAP IV amplifies production of small interfering RNAs (siRNA) that are responsible for methylating cytosine bases in DNA; this methylation silences segments of the genetic code, which can still be transcribed into mRNA but not translated into protein.^{[3][12]} RNAP IV is involved in setting the methylation patterns in the 5S genes during plant maturation, resulting in the development of adult features in plants.

In the first step of heterochromatin formation, RNAP IV couples with an RNA-dependent RNA polymerase known as RDR2 to make a double-stranded precursor to siRNA. Next, DICER-Like protein 3 (DCL3), an enzyme which slices double-stranded RNA substrates, cleaves the double-stranded precursor into siRNAs that are each 24 nucleotides long. These siRNAs are then methylated at their 3′ ends by a protein known as HUA ENHANCER 1 (HEN1). Finally, these methylated siRNAs complex with a protein known as ARGONAUTE-4 (AGO4) in order to form the silencing complex that can perform the required methylation for heterochromatin production. This process is referred to as RNA-directed DNA Methylation (RdDM) or Pol IV-mediated silencing as the introduction of these methyl groups by siRNAs silence both transposons and repetitive sequences of DNA.

SAWADEE HOMEODOMAIN HOMOLOG 1 (SHH1) is a protein that interacts with RNAP IV and is critical in its regulation through methylation. SHH1 can only bind to chromatin at specified “marked” segments, as its “SAWADEE” domain is a chromatin binding domain that probes for unmethylated K4 and methylated K9 modifications on the histone 3 (H3) tail of chromatin; its binding pockets then attach to chromatin at these sites and allow RNAP IV occupancy at these same loci. In this manner, SHH1 functions to enable RNAP IV recruitment and stability at the most actively targeted genomic loci in RdDM in order to promote the previously mentioned siRNA biogenesis of 24 nucleotide-long siRNA. Furthermore, it binds to repressive histone modifications, and any mutations that interfere with this process are associated with a reduction in DNA methylation and siRNA production. Regulation of siRNA production by RNAP IV through this mechanism results in major downstream effects, as the siRNAs produced in this manner defend the genome against the proliferation of invading viruses and endogenous transposable elements.

Homologs of Arabidopsis Pol IV have been identified in soybean, canola, maize, rice, wheat, oat and barley:

| Plant Species | Gene Locus |
|---|---|
| Soybean (Glycine max) | Glyma11g02920.1, |
| | Phy0028Q7_SOYBN |
| Soybean (Glycine max) | Glyma01g42480.1, |
| | Phy00287DG_SOYBN |
| Canola (Brassica napa) | LOC106406243 |
| Canola (Brassica napa) | LOC106420995 |
| Canola (Brassica napa) | LOC106346632 |
| Maize (Zea mays) | COJR65, |
| | GRMZM2G007681_P01, |
| | Phy003UCGF_MAIZE |
| Rice (Orya sativa indica) | B8AT49, |
| | Phy003VB41_ORYSI, |
| | BGIOSGA016952-PA |
| Rice (Orya sativa indica) | B8BED0, |
| | Phy003VIOA_ORYSI, |
| | GBIOSGA031240-PA |
| Rice (Orya glamberrima) | ORGLA05G0052600 |
| Rice (Orya glamberrima) | ORGLA06G0044300 |
| Wheat (T. aestivum) | traesCS7B02G286500 |
| Wheat (T. durum) | TRITD1Bv1G106360 |
| Oats (Avena sativa) | CTG14711 |
| Oats (Avena sativa) | CTG10456 |
| Barley (Hordeum vulgare) | HORVU1Hr1G031780 |
| Barley (Hordeum vulgare) | HORVU2Hr1G113940 |
| Barley (Hordeum vulgare) | HORVU2Hr1G126170 |

D. RNA Pol V

RNA polymerase V (RNA Polymerase V), previously known as RNA polymerase IVb, is a multisubunit plant specific RNA polymerase. It is required for normal function and biogenesis of small interfering RNA (siRNA). Together with RNA polymerase IV (Pol IV), Pol V is involved in an siRNA-dependent epigenetic pathway known as RNA-directed DNA methylation (RdDM), which establishes and maintains heterochromatic silencing in plants. RNA Pol V protein and mRNA accession numbers are AT2G40030 (TAIR Accession: 1009113841; UniProtKB: Q5D869-1; GenPept: 79571777) and AT2G40030A (TAIR Accession: 1009036789 and 4010735536: GenBank Accession: NM_129561), respectively.

RNA polymerase V is composed of 12 subunits that are paralogous to RNA polymerase II (Pol II) subunits. Approximately half of these subunits are shared among Pol II, IV, and V.^{[3]} Its two largest subunits, together forming the catalytic site, make up the most conserved region sharing similarity with eukaryotic and bacterial polymerases. The subunits unique to only Pol IV and V are believed to have arisen from gene duplication events that occurred prior to the evolution of land plants. The structure of Pol V has been studied in a variety of plants, including Arabidopsis thaliana, maize, and cauliflower. Affinity purification has shown significant differences in Pol V composition among these different species.

In Arabidopsis, the largest subunit is known as NRPE1. This subunit contains a GW-rich AGO-hook motif that provides the ability to interact with the argonaute protein AGO4, as well as targeting of DNA methylation. While the subunit is unique to Pol V, it does contain a conserved domain common with the largest subunit of Pol IV known as Defective Chloroplasts and Leaves (DeCL), which provides an unknown function. The second largest subunit of Pol V, NRPD/E2, is shared with Pol IV. Aside from its catalytic site, Arabidopsis Pol V contains 10 smaller, noncatalytic subunits. Of these, 6 are shared with Pol II and 8 are shared with Pol IV. The fourth and seventh subunits form what is known as the “Stalk” subcomplex, while the fifth and ninth subunits form the “Jaw” subcomplex.

Pol V transcribes one of the two types of non-coding RNA involved in RdDM. In canonical RdDM, Pol V transcribes a scaffold RNA which base pairs with a 24-nt siRNA bound to AGO4. The AGO-hook motif in Pol V's largest subunit recruits this AGO4 to the site. Pol V transcripts are also necessary for the recruitment of chromatin remodelers to the target site. One such protein is Domains Rearranged Methyltransferase 2 (DRM2), which is believed to be recruited when the AGO4-bound siRNA base pairs with the scaffold. Once proteins are bound to this scaffold RNA, histone modification and DNA methylation may proceed.

Homologs of Arabidopsis Pol V have been identified in soybean, canola, rice, wheat, oat and barley:

| Plant Species | Gene Locus |
|---|---|
| Soybean (Glycine max) | Glyma13g26690.1, |
| | Phy0028T2C_SOYBN |
| Soybean (Glycine max) | Glyma15g37710.1, |
| | Phy0028X5A_SOYBN |
| Canola (Brassica napa) | LOC106420756 |
| Canola (Brassica napa) | LOC106438049 |
| Canola (Brassica napa) | LOC1-6441872 |
| Rice (Orya sativa indica) | B8A9M1, |
| | Phy003VDXR_ORYSI, |
| | BGIOSGA000052-PA |
| Rice (Orya sativa indica) | B8AHU1, |
| | Phy003BQUC_ORYSI, |
| | BGIOSGA007122-PA |
| Rice (Orya sativa japonica) | B9F2S6, |
| | Phy003SERO_ORYSJ, |
| | LOC_Os02g05880.1, |
| | LOC_Os01g73430.01, |
| | Phy003SKNX_ORYSJ |
| Rice (Orya glaberrima) | ORGLA01G0360200 |
| Rice (Orya glaberrima) | ORGLA11G0102400 |
| Wheat (T. aestivum) | TraesCS4B02G249600 |
| Wheat (T. durum) | TRITD1Av1G222590 |
| Wheat (T. durum) | TRITD4BvG150320 |
| Oats (Avena sativa) | CTG24868 |
| Barley (Hordeum vulgare) | HORVU5Hr1G090000 |
| Barley (Hordeum vulgare) | HORVU5Hr1G089980 |
| Barley (Hordeum vulgare) | HORVU4Hr1G033090 |
| Barley (Hordeum vulgare) | HORVU2Hr1G047470 |

E. Other Proteins

Plant genomes encode for dicer like proteins with similar functions and protein domains as animal and insect dicer. For example, in Arabidopsis thaliana, four Dicer like proteins are made and are designated DCL1 to DCL4. DCL1 is involved with miRNA generation and sRNA production from inverted repeats. DCL2 creates siRNA from cis-acting antisense transcripts which aid in viral immunity and defense. DCL3 generates siRNA which aids in chromatin modification and DCL4 is involved in trans-acting siRNA metabolism and transcript silencing at the post-transcriptional level. Additionally, DCL1 and DCL3 are important for Arabidopsis flowering. In Arabidopsis, DCL knockout does not cause severe developmental problems. DCL3 is proposed as another target gene according to the present disclosure.

Arabidopsis AGOG (Protein Argonaute 6) is involved in transcriptional gene silencing (TGS) and is a component of the RISC complex that associate with the small interfering RNA (siRNA) pathway involved in direct cytosine methylation at endogenous DNA repeats. It appears to be required for the accumulation of specific siRNAs derived from transgene and heterochromatin-related endogenous loci. It is involved in RNA-directed DNA methylation (RdDM) at specific endogenous loci but probably not required for the accumulation of siRNAs derived from transgene inverted repeats that induce post-transcriptional gene silencing (PTGS). It mainly associates with small RNAs of 24 nucleotides in length and preferentially recruits small RNAs with a 5′ terminal adenosine.

Arabidopsis CMT3 is involved in the CpXpG methylation and in gene silencing. Methylates preferentially transposon-related sequences. It is functionally redundant to DRM1/DRM2 to maintain non-CpG methylation. It is involved in RNA-directed DNA methylation. It functions in chromatin binding, DNA (cytosine-5-)-methyltransferase activity and DNA binding.

Arabidopsis DRM2 is involved in RNA-directed DNA methylation (RdDM), acts as major DNA methyltransferase in the RdDM pathway, and is essential for RNA-directed de novo DNA methylation of cytosines in all sequence contexts. It associates with long non-coding RNA (lncRNA) produced by RNA polymerase V (Pol V) and this association is dependent on AGO4 and IDN2, and results in DNA methylation of RdDM target loci.

### III. MODIFYING PLANT GENE EXPRESSION

The present inventors have identified a number of target genes for which modulating the activity or expression of at least one such target gene results in a change in a see characteristic such as seed size, seed number or seed yield. The modulation may be inducing an increase or decrease in target gene expression of target gene product activity. The modulation may also be applied simultaneously to multiple target genes/gene products, such as 2, 3, 4, 5, 6, 7, 8, 9, 10 or more genes/gene products.

The “expression” of a gene or gene product refers to the level of transcription, translation, or protein expression. The “activity” of a gene product relates to its functional capability which may arise from decreased/increased amounts of the gene product or a change in the inherent functionality of an individual molecule. The functional change could be change in enzyme activity, cellular localization, or interaction with other proteins or nucleic acids, etc.

Measurement of the level or amount of a gene product may be carried out by any suitable method, for example including comparison of mRNA transcript levels, protein or peptide levels, and/or phenotype of a plant, between a modified plant and comparable plant which has not been modified according to the present disclosure.

The term “a comparable product” as defined herein would be one derived from a plant which had not been modified according to the present disclosure but in which all other relevant features were the same (e.g., plant species, growing conditions, method of processing the plant). The comparable product according to the present disclosure may mean a plant or a part thereof, such as a seed, a harvested seed, a processed seed leaf or seed material or combinations thereof obtainable or obtained from a plant which has not been modified in accordance with the present disclosure, i.e., to modulate the activity or expression of a target gene or gene product. In an embodiment, a comparable product is one which does not comprise a functional gene whose activity or expression has been modulated.

The term “modifying” or “modified” as used herein means a plant that has been altered or changed. The present disclosure comprises the modification of plants using techniques for genetic modification of plants or non-genetic modification of plants. Such methods are well known in the art and examples of genetic modification techniques include transformation, transgenics, cisgenics, and gene editing methods. Examples of non-genetic modification techniques include fast-neutron mutagenesis, chemical mutagenesis, e.g., ethyl methanesulfonate (EMS) mutagenesis and modern population analysis approaches.

The term “unmodified plant” as defined herein would be a plant which had not been modified according to the present disclosure, to modulate the activity or expression of a protein kinase gene and in which all other relevant features were the same. In an embodiment, an unmodified plant is one which does not comprise a functional gene whose activity or expression has been modulated.

The term “increasing” (e.g., increasing the activity or expression of a gene or gene product) as used herein means that the activity or expression higher compared with the activity or expression of the gene or gene product in an unmodified comparable plant.

In some embodiment, a modification which increases the activity or expression is selected from increasing, promoting or augmenting transcription, translation or expression; increasing synthesis of the target gene product or its release from intracellular stores; or decreasing the rate of degradation of the gene product. The activity of a gene product may be increased by introducing a construct comprising a strong promoter and a nucleotide sequence encoding a gene product as described herein. Alternatively, the endogenous promoter of a gene product may be modified in such a way that it is more active in the modified plant than in an unmodified plant. Means for modifying an endogenous promoter can be, for example, gene editing methods, CRISPR Cas9, TALENS or zinc finger nucleases. Alternatively, additional copies of the gene encoding the gene product including its natural promoter may be incorporated into the plant.

In embodiments, the expression of at least one target gene product may be increased by at least about 1%, at least about 3%, at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 1000%, at least about 2500%, or at least about 5000% more expression compared to an unmodified gene product/plant cell.

The methods may comprise transforming a cell of a plant with a genetic construct which encodes a target gene product or a functional variant or functional fragment or orthologue thereof. It will be appreciated that each of these options would result in an increased activity and expression of the target gene product. The method may comprise regenerating the plant from the transformed cell. There is also provided use of genetic construct which is capable of increasing the activity and/or expression of a target gene product in a plant transformed with the construct.

In another embodiment, the activity of at least one target gene product may be increased by introducing (or providing) a mutation to the corresponding target gene. In an embodiment, the modified gene product may exhibit increased activity compared to an unmodified gene product, such at least about 1%, at least about 3%, at least about 5%, at least about 10%, at least about 20%, at least about 30%, at least about 40%, at least about 50%, at least about 60%, at least about 70%, at least about 80% or at least about 90% more activity compared to an unmodified gene product/plant cell. In another embodiment, the modified gene product comprises a mutation which renders the gene product constitutively active. In other words, the gene product continuously functions.

The expression of specific gene products can be measured by measuring transcription and/or translation of the gene. Methods for measuring transcription are well known in the art and include, amongst others, northern blot, RNA-Seq, in situ hybridization, DNA microarrays and RT-PCR. Alternatively, the expression of a gene may be measured indirectly by measuring the level of the gene product for example the protein encoded by said gene.

The term “inhibiting” (e.g., inhibiting the activity or expression of a gene product) as used herein means that the activity or expression of the gene product is lower or decreased compared with the gene activity or expression of the gene in a comparable product. Thus, the expression or activity of the gene product may be reduced, partly inactivated, inhibited, eliminated, knocked out or lost such that the protein expression or function of the gene product is not detectable. In one aspect, the target gene is knocked out. In other words, the target gene has been rendered completely inoperative.

Examples of reducing expression include providing a mutation in a regulatory region (e.g., a promoter or an enhancer) which contributes to controlling the expression of a gene product, or providing an antisense RNA, siRNA or miRNA which reduces the level of nucleic acid sequence encoding a gene product. In another embodiment, the activity of at least one target gene product may be decreased by introducing (or providing) a mutation to the corresponding target gene. In an embodiment, the modified gene product may exhibit decreased activity or expression compared to an unmodified gene product/plant cell, such at least about 1%, at least about 3%, at least about 5%, at least about 10%, at least about 20%, at least about 30%, at least about 40%, at least about 50%, at least about 60%, at least about 70%, at least about 80% or at least about 90% less activity or expression compared to an unmodified gene product/plant cell.

Any method known in the art for reducing or preventing the expression or activity of a protein may be used in the methods according to the present disclosure. By way of example, the present methods may comprise providing one or more mutations in a nucleic acid sequence which encodes a gene product. As used herein, the term “mutation” encompasses a natural genetic variant or an engineered variant. In particular, the term “mutation” refers to a variation in the nucleotide sequence encoding the target gene product.

In an embodiment, a method according to the present disclosure may comprise providing a nucleic acid sequence to a plant or part thereof or plant cell, wherein said nucleic acid results in the reduction or elimination of the expression or activity of a protein kinase gene. Suitably said nucleic acid sequence may be introduced to the plant or part thereof or cell. Suitably an endogenous nucleic acid sequence in the plant or part thereof or cell may be modified to encode the polypeptide according to the present disclosure (e.g., by gene editing).

In a particular embodiment, the plant or plant cell according to the present disclosure is homozygous. Suitably, the plant or plant cell may be homozygous for the modification e.g., inhibition or mutation.

In an embodiment, the plant or plant cell according to the present disclosure expresses only the modified e.g., mutated nucleic acid encoding the gene product. In other words, in some embodiments no endogenous (or endogenous and functional protein) is present in the plant according to the present disclosure. In other words, if any endogenous protein is present it is preferably in a reduced or inactive form.

In an embodiment, an interruption may cause the nucleic acid sequence to not be transcribed and/or translated. The nucleic acid sequence may be interrupted, for example, by deleting or otherwise modifying the ATG start codon of the nucleic acid sequence such that translation of the protein is reduced or prevented. The nucleic acid sequence also may comprise one or more nucleotide change(s) that reduce or prevent expression of the protein or affect protein trafficking. For example, expression of the protein may be reduced or prevented by introduction of one or more pre-mature stop codons, a frame shift, a splice mutant or a non-tolerated amino acid substitution in the open reading frame. A premature stop codon refers to a mutation which introduces a stop codon into the open reading frame and prevents translation of the entire amino acid sequence. The premature stop codon may be a TAG (“amber”), TAA (“ochre”), or TGA (“opal” or “umber”) codon.

A frame-shift mutation (also called a framing error or a reading frame shift) is a mutation caused by indels (insertions or deletions) of a number of nucleotides in a nucleic acid sequence that is not divisible by three. Due to the triplet nature of gene expression by codons, the insertion or deletion can change the reading frame, resulting in a completely different translation from the original. A frameshift mutation will often cause the reading of the codons after the mutation to code for different amino acids. The frameshift mutation will commonly result in the introduction of a premature stop codon.

Splice mutant inserts, deletes or changes of a number of nucleotides in the specific site at which splicing takes place during the processing of precursor messenger RNA into mature messenger RNA. The deletion of the splicing site results in one or more introns remaining in mature mRNA and may lead to the production of abnormal proteins.

A non-tolerated amino acid substitution refers to a mutation which causes a non-synonymous amino acid substitution in the protein which results in reduced or ablated function of the protein. Any method known in the art for providing a mutation in a nucleic acid sequence may be used in the method according to the present disclosure. For example, homologous recombination may be used, in which a vector is created in which the relevant nucleic acid sequence(s) are mutated and used to transform plants or plant cells. Recombinant plants or plant cells expressing the mutated sequence may then be selected.

In an embodiment, the nucleic acid sequence may be wholly or partially deleted. The deletion may be continuous or may comprise a plurality of sections of sequence. The deletion preferably removes a sufficient amount of nucleotide sequence such that the nucleic acid sequence no longer encodes a functional protein. The deletion may be total, in which case 100% of the coding portion of the nucleic acid sequence is absent, when compared to the corresponding genome of a comparable unmodified plant. The deletion may, for example, remove at least 50, 60, 70, 80 or 90% of the coding portion of the nucleic acid sequence. Suitably, at least part of the protein may be deleted. The deletion may, for example, remove at least 10, 20, 30, 40, 50, 60, 70, 80 or 90% of the coding portion of the protein. Suitably, the deletion may remove at least 50 amino acids, at least 100 amino acids, at least 150, at least 200, at least 250, amino acids from the gene product. In an embodiment, the deletion may remove at least 100 amino acids, at least 150, at least 200, at least 250, at least 300, at least 350 amino acids from the C terminus of the gene product.

Methods for deletion of nucleic acid sequences in plants are known in the art. For example, homologous recombination may be used, in which a vector is created in which the relevant nucleic acid sequence(s) are missing and used to transform plants or plant cells. Recombinant plants or plant cells expressing the new portion of sequence may then be selected.

Plant cells transformed with a vector as described herein may be grown and maintained in accordance with well-known tissue culturing methods such as by culturing the cells in a suitable culture medium supplied with the necessary growth factors such as amino acids, plant hormones, vitamins, etc.

Modification of the nucleic acid sequence may be performed using targeted mutagenesis methods (also referred to as targeted nucleotide exchange (TNE) or oligo-directed mutagenesis (ODM)). Targeted mutagenesis methods include, without limitation, those employing zinc finger nucleases, TALENs (see WO2011/072246 and WO2010/079430), Cas9-like, Cas9/crRNA/tracrRNA, Cas9/gRNA, or other CRISPR systems (see WO 2014/071006 and WO2014/093622), meganucleases (see WO2007/047859 and WO2009/059195), or targeted mutagenesis methods employing mutagenic oligonucleotides, possibly containing chemically modified nucleotides for enhancing mutagenesis with sequence complementarity to the gene, into plant protoplasts (e.g., KeyBase® or TALENs).

Alternatively, mutagenesis systems such as TILLING (Targeting Induced Local Lesions IN Genomics; McCallum et al., 2000, Nat Biotech 18:455, and McCallum et al., 2000, Plant Physiol. 123, 439-442, both incorporated herein by reference) may be used to generate plant lines which comprise a gene encoding a protein having a mutation. TILLING uses traditional chemical mutagenesis (e.g., ethyl methanesulfonate (EMS) mutagenesis, which produces random mutations) followed by high-throughput screening for mutations. Thus, plants, seeds, cells and tissues comprising a gene having the desired mutation may be obtained.

The method may comprise the steps of mutagenizing plant seeds (e.g., EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such modified plants. Seeds may, for example, be radiated or chemically treated and the plants may be screened for a modified phenotype.

Fast neutron deletion mutagenesis may be used in a reverse genetics sense (i.e. with PCR) to identify plant lines carrying a deletion in the endogenous gene. See for example Ohshima et al. (1998) Virology 213:472-481; Okubara et al. (1994) Genetics 137:867-874; and Quesada et al. (2000) Genetics 154:421-4315 which are incorporated herein by reference.

In another approach, dominant mutants may be used to trigger RNA silencing due to gene inversion and recombination of a duplicated gene locus. See for example Kusaba et al. (2003) Plant Cell 15:1455-1467 (incorporated herein by reference).

Modified plants may be distinguished from non-modified plants, i.e., wild-type plants, by molecular methods, such as the mutation(s) present in the DNA, and by the modified phenotypic characteristics. The modified plants may be homozygous or heterozygous for the modification. Preferably modified plants are homozygous for the modification.

The method of reducing or preventing the expression of a gene or gene product may comprise treating the plant with a chemical (e.g., an agrochemical), use of virus-induced gene silencing (VIGs), micro RNA silencing, RNAi, antisense suppression, sense suppression, tDNA insertions, targeted mutagenesis, dominant negative constructs (or antimorphic mutations), a CRISPR-based system or by zinc finger nuclease, TALENs, meganucleases, mutagenic oligonucleotides or TILLING.

### IV. PLANT PROPAGATION, BREEDING AND SEED HARVEST

In one embodiment, the disclosure provides a method of producing a plant having modified expression and/or activity of a selected gene product. The method crossing a donor plant having modified expression and/or activity with a recipient tobacco plant that does not have modified expression/and or activity and possesses commercially desirable traits. Genetic material is then isolated from a progeny of said donor plant crossed with said recipient plant. Analysis of the genetic material can then be performed to identifying an introgressed region comprising a mutation or a heterologous polynucleotide sequence. Plants may subsequently be inbred, outbred and/or subjected to seed harvesting.

### V. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Plant Growth Conditions. Arabidopsis thaliana plants were grown on soil with 3-day cold treatment before moving into growth chambers under 16-hour days/8-hour nights with temperatures of 23° C. during the day and 22° C. at night. Arabidopsis plants that were geminated on MS (Murashige and Skoog) media plates were first sterilized. Seeds were aliquoted into 1.5 ml Eppendorf tubes. 70% ethanol was added into the tubes, tubes were vortexed for several seconds, seeds were pelleted, and then ethanol was removed. Seeds were washed in 1 ml 50% Bleach for 10 minutes with vortexing. Then, seeds were washed with diH₂O for three to four times. Appropriate amount of 0.1% agarose was added to the sterilized seeds. The seeds were spread evenly onto the MS media plates that contained the appropriate selection. The MS media plates were placed at 4° C. for three days before being moved into growth chamber.

Arabidopsis Crosses. Arabidopsis young flower buds at stage 13 were used as female plant for crossing. Siliques, opened flowers, and flower buds younger than stage 13 on the same flowering stalk were removed. Forceps were sterilized with 95% ethanol before and after dissecting each flower bud. Flower buds at stage 13 were dissected under stereo microscope. Sepals, petals, and stamens were removed with forceps carefully. During dissecting the flower bud, the pistil and flower stalk should not get injured. From another Arabidopsis plant which was used as male plant, an opened flower with mature pollen grains was picked. The mature pollen grains were used to pollinate the pistil of previously dissected flower bud. Pollen from several anthers were used to pollinate the same pistil to increase the chance of pollination. After pollination, the pistil was coved with a plastic bag to avoid contacting with other undesired pollen. The cross was examined after 1 or 2 days to make sure it was successful. The pistil would elongate after pollination. If the pollination was not successful, a second-round pollination should be conducted. The plastic bag was removed a few days after crossing. The crossed plants were used based on different demands.

Endosperm and Embryo dissection/FACS staining and sorting Col-0 and arf8-2 plants were emasculated and roughly 24 hours later the plants were pollinated. 9 days later seeds were dissected to remove endosperm and embryo into two different tubes filled with 140 μl of nuclei extraction buffer. Nuclei extraction buffer contains 120 μl of Partec Cystain UV precise P Nuclei extraction buffer (Sysmex America #05-5002). 20 μl of Protease Inhibitor 7× stock solution One complete mini EDTA-free Protease inhibitor Cocktail Tablets (Roche #11836170001) in 1.5 ml diH₂O. 200 seeds for each crosses were dissected so that each cross (WT×WT, arf8-2×arf8-2, WT×arf8-2, and arf8-2×WT) contained 200 embryos and 200 endosperm. Dissection is performed by placing a silique on double sided tape and then removal of seeds from siliques. An incision is performed on the seed to remove the embryo. The endosperm is dissection by using the tape to collect the seed coat contamination.

The day after embryo and endosperm must be properly stained. Preparation of 800 μl of staining buffer is needed for each sample. 686 μl of Nuclei Staining buffer (Staining buffer from Partec CyStain UV Precise P Sysmex America #05-5002 with 1/7 total volume of 7×Protease Inhibitor (114 μl). First prepare the needed amounts of nuclei staining needed (800 μl per sample) and then wrap the Eppendorf tube with aluminum foil to protect the buffer from light. Keep the buffer and sample on ice. On ice grind the sample in the nuclei extraction buffer with a pestle for 2-3 minutes for each sample. Then add 800 μl of the Nuclei staining buffer with the Protease Inhibitor. Pipette up and down about ten times to mix the staining buffer with the sample. Filter the sample in the extraction and nuclei staining buffer twice with CellTrics 30 um filter (Partec #04-0042-2316) on ice. Sort in the flow cytometry machine (BD FACSAria). Use 1.5 ml tubes containing 50 μl CTAB to collect the sorted nuclei.

Whole-Mount Seed Clearing and Microscopy. Whole-mount immature seeds were dissected from both WT Col-0 and arf8-2 siliques 1 to 8 DAP, cleared in Hoyer's fluid (70% (w/v) chloral hydrate, 4% (v/v) glycerol, and 5% (v/v) gum arabic), and observed with a Leica SP6 Epifluorescence microscope using differential interference contrast optics in the Department of Pathology of the Medical School at Saint Louis University.

Gene Expression Analysis. To examine the expression of AP2, ANT, CYCD3:1, ACTIN, and GAPDH in the arf8-2 mutant allele, ydk1 mutant allele and WT Col-O, (arf8-2/arf8-2), (ydk1/ydk1), and WT Col-0 seeds at 3 DAP, 7DAP, 21 DAP from each plant was isolated, and total RNA was extracted. Gene expression was examined by using semiquantitative RT-PCR using 30 PCR amplification cycles or quantitative RT-PCR. First-strand cDNA was synthesized using the ProtoScript First Strand cDNA Synthesis Kit M-MuLV Reverse Transcriptase (New England Biolabs M0253L). PCR product was amplified, and ACTIN was used as an internal control.

Quantitative RT-PCR was performed using QuantStudioR 5 Real-Time PCR Instrument and applied biosystems Software (Thermo Fisher Scientific). The primer pairs for amplifying AP2 (At4g36920) in arf8-2, ydk1 and WT Col-0 cDNA were as follows:

forward
(SEQ ID NO: 1)
5′-CGCGGGTAAAGCTACCAA-3′,
and
reverse
(SEQ ID NO: 2)
5′-CTAAGTAAACTTGTTTCCCACAGTC-3′

The primer pairs for amplifying ANT (At4g37750) in arf8-2, ydk1 and WT Col-0 cDNA were as follows:

forward
(SEQ ID NO: 3)
5′-GCTCTCAACAGATCTTCTCACTC-3′,
and
reverse
(SEQ ID NO: 4)
5′-TCTGGTTTGAGGGAAGCTAAAG-3′.

The primer pairs for amplifying CYCD3: 1(At4g34160) in arf8-2, ydk1 and WT Col-0 cDNA were as follows:

forward
(SEQ ID NO: 5)
5′-CTGTCGCGTGTCTCTCATTA-3′,
and
reverse
(SEQ ID NO: 6)
5′-TCCACTCGAGAGTAGACAGAA-3′.

The primer pairs for amplifying GAPDH (At3g26650) in arf8-2, ydk1 and WT Col-0 cDNA were as follows:

forward
(SEQ ID NO: 7)
5′-CTAACCGAAACCCGTCTCTTC-3′,
and
reverse
(SEQ ID NO: 8)
5′-GTTTCCCTGCACCTTCTCTATC-3′.

ARF8 Complementation and Overexpression Cloning. The ARF8 promoter and genomic DNA region was PCR amplified with primers ARF8 promoter and gDNA forward1 (5′-CGCGGTCGACTCTCCAAGTGATACACTCCATATCC-3′ (SEQ ID NO: 9) and ARF8 promoter and gDNA reverse1 (5′-CGCGGTCGACGAGATGGGTCGGGTTTTGCGGGAAT-3′ (SEQ ID NO: 10)). The SalI cut site is underlined for easy viewing. The amplified ARF8 promoter and genomic DNA region were cloned into vector pBI-EGFP. The constructs were confirmed by sequencing and transformed into Arabidopsis Col-0 by Agrobacterium infiltration. After screening for transgenic plants, stable transgenic T2 or later plants were used for to measure its weight, length, and width.

The ARF8 genomic DNA region was PCR amplified with primers ARF8 gDNA forward1 (5′-CGCGGTCGACATGGAACGAAACGAGAAAACAAGTC-3′ (SEQ ID NO: 11)) and ARF8 gDNA reverse1 (5′-CGCGGGATCCGAGATGGGTCGGGTTTTGCGGGAAT-3′ (SEQ ID NO: 12)). The SalI cut site for ARF8 gDNA foward1 is underlined as well as the BamHI cut site for ARF8 gDNA reverse1. The amplified ARF8 DNA region was cloned into vector pBI-121 with a 35s promoter. The constructs were confirmed by sequencing and transformed into Arabidopsis Col-0 by Agrobacterium infiltration. After screening for transgenic plants, stable transgenic T2 or later plants were used for to measure its weight, length, and width.

Measuring Seed Weight, Length, and Width. Mature F1 dry seeds in batches of 500 were weighed using an analytical balance. (Mettler Toledo AB54-S/FACT). Three replicates were done for all crosses. For measuring seed length and width, photos of seed population of were taken under a microscope (Leica MZ 16F microscope), then seed length and width were measured using Q-capture pro system. 200 seeds from each cross were measured for seed size. Arabidopsis Mutant Seed Generation

T-DNA mutant lines of ARF8 gene (ARF8-2) were purchased from Arabidopsis Biological Resource Center (ABRC). The T-DNA line of ARF8-2 (AT5G37020) is arf8-2 (CS24608). To identify T-DNA insertion in arf8-2 mutants, primers ARF8 reverse1 (5′-GACGCTTGTTGCTGCTGTTG-3′ (SEQ ID NO: 13)) and LB1 (5′-GCTTCCTATTATATCTTCCCAAATTAC-3′ (SEQ ID NO: 14)) were used to amplify a 390 bp fragment in arf8-2 mutants. To amplify the endogenous gene region across the T-DNA insertion, primers ARF8 forward1 (5′-TGGGAATTCGTCATGCCACTC-3′ (SEQ ID NO: 15)) and ARF8 reverse1 (5′-GACGCTTGTTGCTGCTGTTG-3′ (SEQ ID NO: 16)) were used to amplify a 1364 bp fragment in ARF8.

Single arf8-2 were back-crossed to wild-type three times. After each back cross, arf8-2 mutants were confirmed to contain the corresponding T-DNA insertion. The arf8-2 mutant are in Col-0 background.

T-DNA mutant lines of YDK1 gene (ydk1) were purchased from Arabidopsis Biological Resource Center (ABRC). The T-DNA line of YDK1 (AT4G37390) is ydk1 (SALK_135479). To identify T-DNA insertion in ydk1 mutant, primers AT4G37390 forward1 (5′-GACGAGGCTGAGCTACAAA-3′ (SEQ ID NO: 17)) and LBa1 (5′ TGGTTCACGTAGTGGGCCATCCG-3′ (SEQ ID NO: 18)) were used to amplify a 980 bp fragment in ydk1 mutants. To amplify the endogenous gene region across the T-DNA insertion, primers AT4G37390 forward1 (5′-GACGAGGCTGAGCTACAAA-3 (SEQ ID NO: 19)′) and AT4G37390 reverse 1 (5′-AGGGCGTAGTAATAATTAAATGCTG-3′ (SEQ ID NO: 20)) were used to amplify a 786 bp fragment in YDK1.

### Example 2—Results and Discussion

A. Results

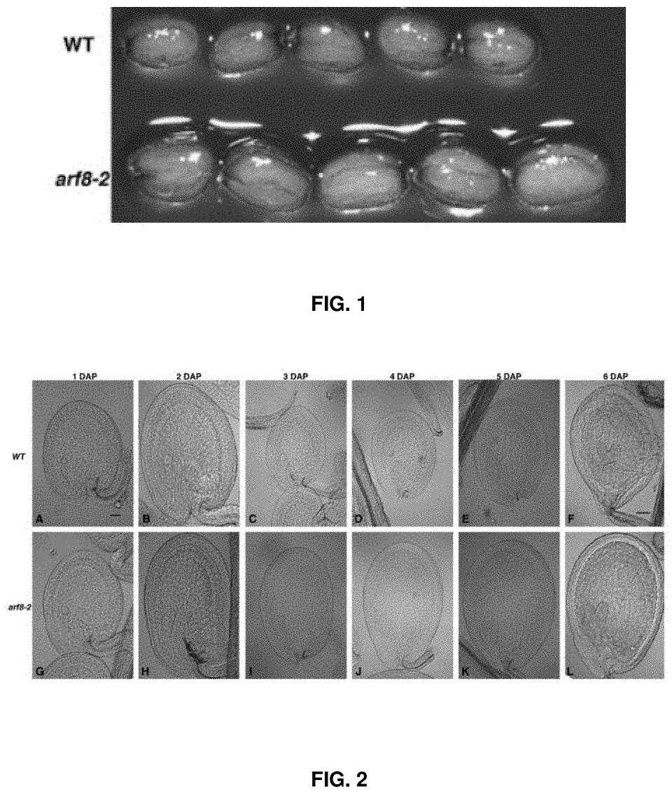

Effects of the arf8-2 mutation on seed weight, length, and width. Seed generated with the arf8-2 homozygous F1 mutant was larger compared to wild-type seed (FIG. 1). To assess quantitatively the effects of ARF8 in regulating seed size, the inventors measured F1 seed weight from the arf8-2 homozygous mutant and WT Col-0. As shown in Table 1, the mutant arf8-2 produced significantly larger seeds than WT Col-0. arf8-2 mutants on average weighed around 23.14 mg per 1000 seeds while wild-type weigh around 18.72 mg. This represents a 23.6% increase in weight. To further test the ARF8 role in regulating seed size, the inventors measured seed length and width of the self-pollinated arf8-2 homozygous mutant and WT Col-0. As shown in Table 1, the mutant arf8-2 produced significantly larger seeds than WT Col-0. The average wild-type seed length was approximately 448 μm while arf8-2 mutants had an average seed length of 506 μm which increased by 12.9%. Measuring the width of wild-type and arf8-2, a similar increase in seed width was detected: arf8-2 had a seed width of 331 μm which was 17.0% wider than the wild-type width of 283 μm.

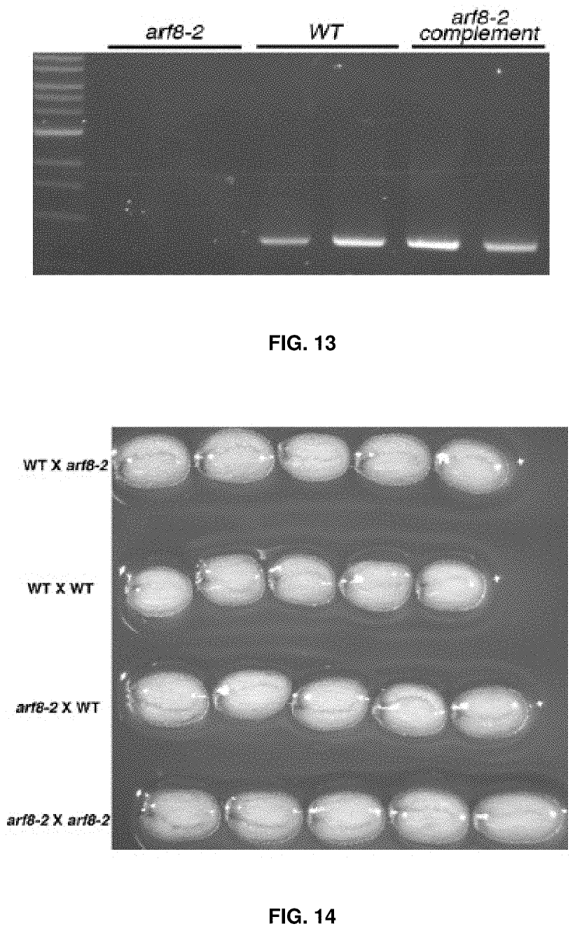

To further confirm that this larger seed size phenotype was caused by the T-DNA insertional mutation in the ARF8 gene, the inventors transformed the ARF8 promoter and coding region into the arf8-2/arf8-2 homozygous plants. They tested ARF8 expression in the arf8-2 homozygous mutant, WT Col-0, and the arf8-2 complement with ARF8 transgene. FIG. 13 showed ARF8 expression by RT-PCR in leaf tissue. It showed that T-DNA insertion in the arf8-2 mutant caused knockout of the gene while arf8-2 complement plant restores ARF8 expression. The result showed that the transgene complemented the mutant phenotype and produce seeds that were much more similar in weight, length, and width to WT Col-0 (Table S1). This indicates that a loss expression of ARF8 is sufficient to increase seed weight, length, and width.

To examine whether the arf8-2 mutant has a parent-of-origin effect on seed size, reciprocal crosses were performed between the arf8-2 mutant an WT Col-0. Crosses of the maternal WT Col-0 pollinated with paternal WT Col-0 and the maternal arf8-2 mutant pollinated with the paternal arf8-2 were performed as controls. As shown in FIG. 14 and Table S2, the seed weight, length, and width were all larger for the homozygous arf8-2 mutant as compared to WT Col-0 seeds. The F1 seeds of the arf8-2 female crossed with the arf8-2 male weighed 28.24 mg per 1000 seeds while the wild-type control cross weighed 21.86 mg per 1000 seeds, representing 29.2% increase. The average seed length of the arf8-2 mutant cross was 563 μm which increased by 11.7% in comparison to the wild-type of 504 μm. The average width of the arf8-2 mutant cross was 331 μm which increased by 8.52% in comparison to the wild-type of 305 μm. Comparing the self-pollinated arf8-2 mutant seeds with the manual cross-pollinated arf8-2 mutant seeds, the out-crossed progeny cause larger, longer, and wider seeds in the cross-pollinated arf8-2 mutant seeds. This was also seen in the cross-pollinated wild-type when compared to their self-pollinated wild-type counterpart in Table 1.

TABLE 1
Effects of the arf8-2 mutation on seed weight, length and width.

| Genotype | Weight^{a} | Change^{b} | Length^{c} | Change^{b} | Width^{c} | Change^{b} |
|---|---|---|---|---|---|---|
| Wild Type | 18.72 ± 0.112 | — | 448 ± 2.00 | — | 283 ± 5.51 | — |
| arf8-2 | 23.14 ± 0.118 | +23.6% | 506 ± 12.10 | +12.9% | 331 ± 9.29 | +17.0% |

^{a}One thousand seeds were weighed. Weight (mg) per 1,000 seeds is shown. Standard deviation for three replicates is shown.
^{b}Change is relative to wild-type seed.
^{c}Average length and width (μm) of 200 seeds is shown.

ARF8 plays a role in endosperm and embryo development. To determine whether the arf8-2 induced effects on seed mass reflected an increase in seed size during early embryogenesis, the inventors isolated and visualized the arf8-2 mutant and wild-type seeds at 1-6 days after pollination (DAP). By using a seed clearing solution, they were able to visualize the seed cavity and the embryo of the mutant and wild-type as the seed develops. The homozygous arf8-2 mutants were longer, wider and had a larger seed cavity than wild-type seeds at every stage examined (FIG. 2).

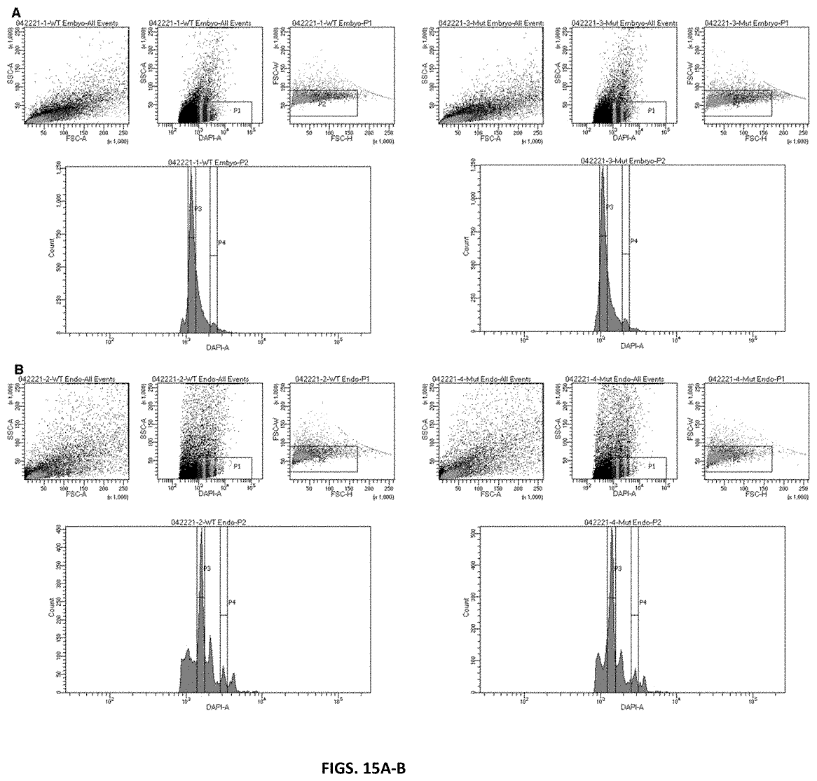

The endosperm and embryo are important tissues in seed. Since ARF8 affects seed size, the inventors speculated if it could affect endosperm and embryo development. By using flow cytometry and fluorescence-activated cell sorting (FACS), the inventors can isolate and distinguish endosperm and embryo nuclei. In an Arabidopsis seed, there are three distinct seed tissues: the diploid embryo and seed coat, and triploid endosperm compromising of two maternal genomes and one paternal genome. DAPI (4′,6-diamidino-2-phenylindole) is a fluorescent dye that binds to AT rich regions of DNA. Staining embryo and endosperm tissues with DAPI can allow separation of embryo and endosperm by different nuclei staining intensity per cell. Removing the embryo and separating the endosperm from the seed coat allows endosperm nuclei to be distinguished from embryo nuclei by different DAPI intensity resulting from different DNA content as shown in FIGS. 15a-b. 2C and 4C peaks represents the diploid embryo while the 3C and 6C peaks represents the endosperm. The 4C and 6C peaks are the embryo and endosperm cells that are undergoing DNA replication. By sorting and collecting the nuclei from those peaks the inventors can roughly estimate how many cells are present in the endosperm and embryo of arf8-2 and WT Col-0 at 9 DAP.

As shown in Table 2, the arf8-2 mutant caused a change in cell number to both the embryo and endosperm. At 9DAP, the inventors saw an increase in the number of embryo cells from the WT Col-0 to the arf8-2 mutant. Two hundred wild-type seeds had 179,296 cells in embryo while arf8-2 had 455,021 cells in embryo which represents an increase of 154%. In addition, the inventors saw an increase in the number of endosperm cells from WT to the arf8-2 mutant. Two hundred wild-type seeds had 39,139 cells in endosperm while arf8-2 produced an endosperm cell number of had 57,236 cells in endosperm which represents 46.2% increase. These results indicate that a loss in expression of the auxin response factor 8 is sufficient to increase the cell number of both the endosperm and embryo during seed development.

TABLE 2
Effects of the arf8 mutation on cell numbers
of embryo and endosperm (200 seeds/replicate)

| | Cell number of | | Cell number of | |
|---|---|---|---|---|
| Genotype | embryo ^{a} | Change^{b} | endosperm ^{a} | Change^{b} |
| Wild type | 179,296 ± 37985 | — | 39,139 ± 16 | — |
| arf8-2 | 455,021 ± 95735 | +154% | 57,236 ± 1098 | +46.2% |

^{a} Two hundred seeds were sorted. Total sorted cell number of embryo or endosperm of 200 seeds is shown. Standard deviation for three replicates is shown.
^{b}Change is relative to wild-type seed.

To assess whether the arf8-2 mutants have a parent-of-origin effect on embryo and endosperm cell numbers, the inventors performed reciprocal crosses, dissected 200 seeds and separated the embryo and endosperm into their own Eppendorf tube and sorted the nuclei of the embryo and endosperm by FACS as stated above. As shown in Table S3, the inventors saw a similar increase of cell numbers: 144.0% and 67.3% increase in embryo and endosperm, respectively, in manual cross-pollinated arf8-2 compared with cross-pollinated WT. The F1 seeds of the maternal WT crossed with the paternal arf8-2 mutant had 425,832 cells in embryo and 64,565 cells in endosperm which represents an increase of 106% from the wild-type control cross of 206,277 cells in the embryo, and an increase of 54% from the wild-type control cross of 42,017 cells in the endosperm. F1 seeds of maternal WT crossed with the paternal arf8-2 had more embryo and endosperm cells than maternal arf8-2 crossed with the paternal WT.

The F1 seeds of the maternal arf8-2 crossed with the paternal WT mutant had 398,819 cells in embryo and 58,084 cells in endosperm which represents an increase of 93% from the wild-type control cross of 206,277 cells in the embryo, and an increase of 38% from the wild-type control cross of 42,017 cells in the endosperm. While F1 seeds of maternal WT crossed with the paternal arf8-2 had more embryo and endosperm cells than maternal arf8-2 crossed with the paternal WT, the differences between them were not statically significant. Having both mutant arf8-2 alleles though did produce seeds with the most amount of embryo and endosperm cells but it seems like having either paternal or maternal mutant alleles was sufficient to produce seeds with more embryo and endosperm cells than WT.

Effects of the ydk1 mutation on seed weight, length, and width. There are 23 ARF in Arabidopsis and some may bind to GRETCHEN HAGEN 3 (GH3)'s promoters to regulate gene expression. ARF8 was the first ARF that was discovered to regulate the expression of three AtGH3 genes: AtGH3a, DWARF IN LIGHT 1(DFL1), and YADOKARI1 (YDK1). The GH3 genes were downregulated in the arf8 mutant and up-regulated in ARF8 overexpression lines suggesting that ARF8 positively regulates expression of GH3 genes. Since arf8-2 was shown to increase seed size in Arabidopsis, the inventors speculated if YDK1, a target of ARF8 transcription factor, could also have an effect on seed size. To assess whether YDK1 plays a role in regulating seed size, they measured seed weight of the ydk1 homozygous mutant and WT Col-0.

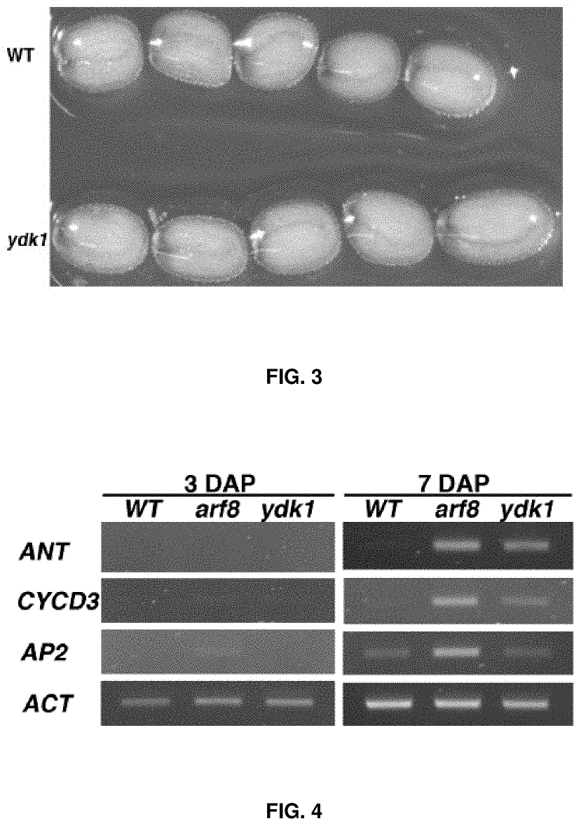

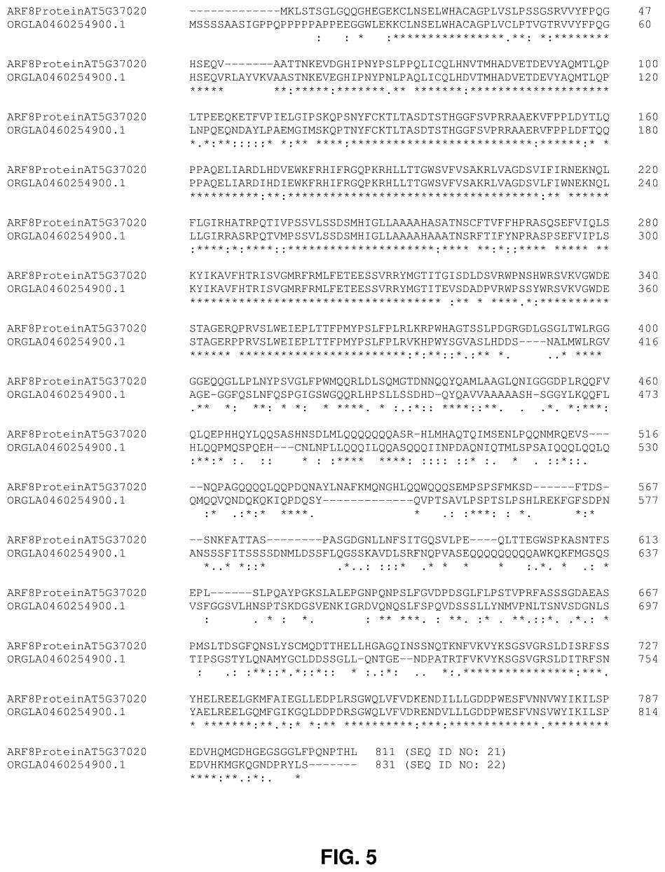

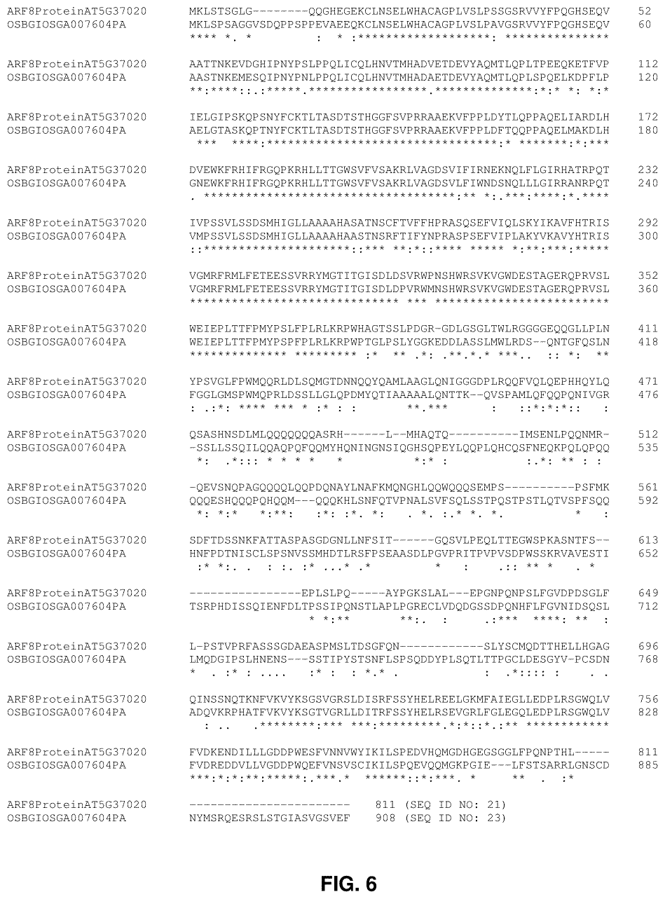

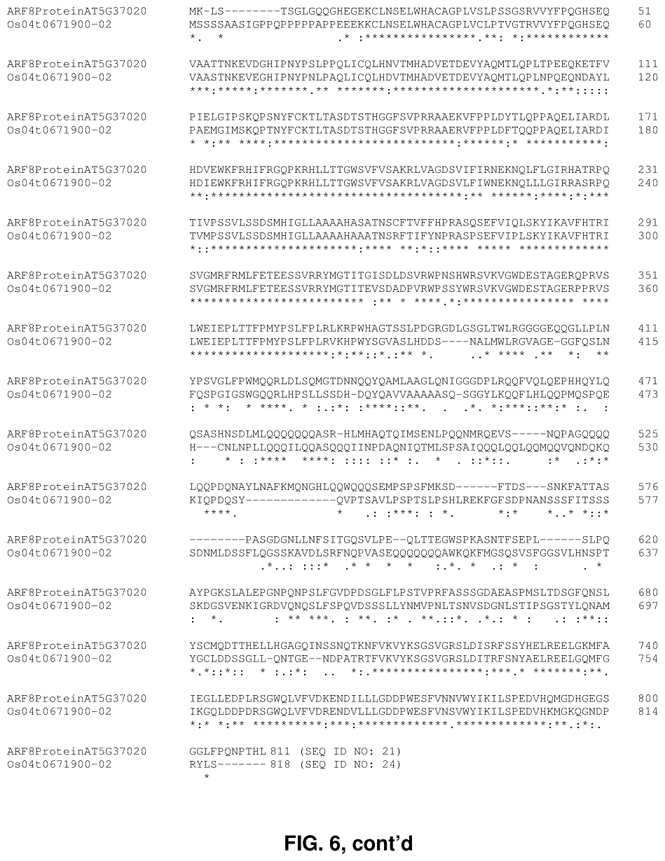

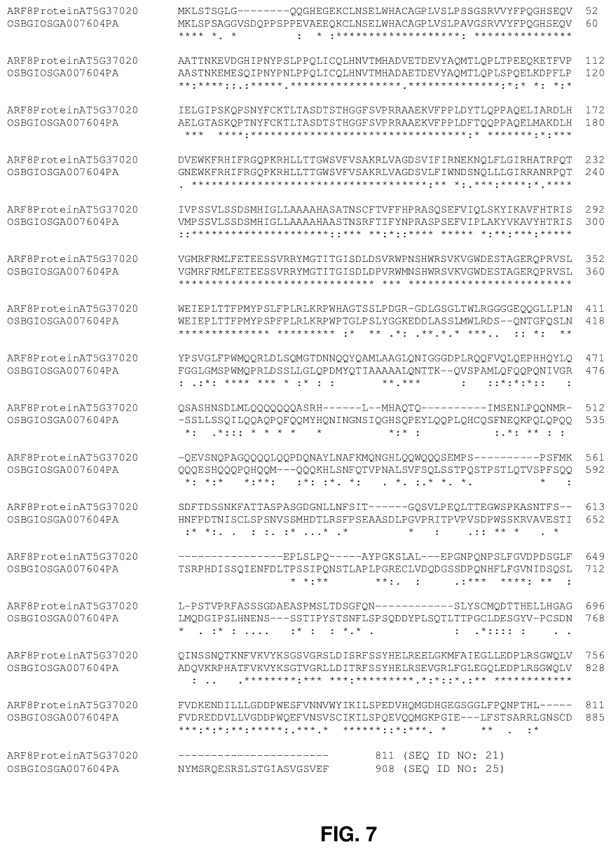

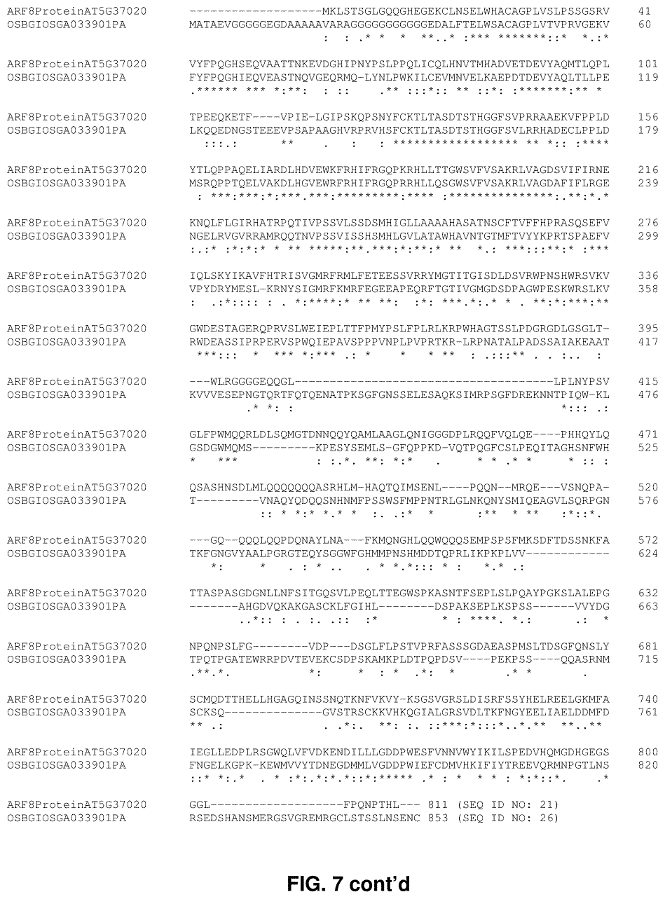

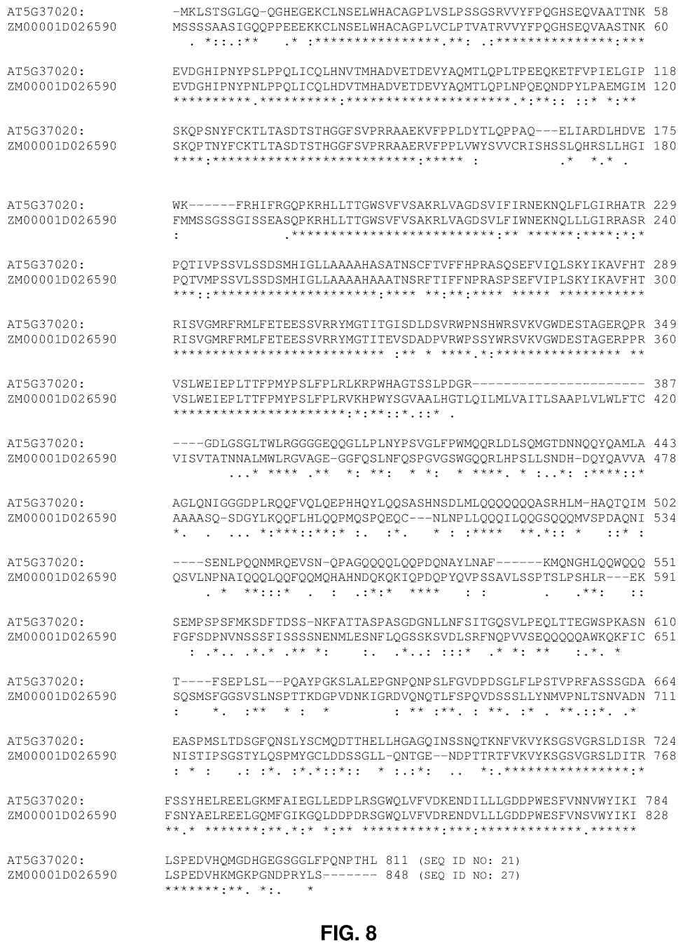

As shown in FIG. 3, the mutant ydk1 produced significantly larger seeds than WT Col-0. The selfed ydk1 seeds had a weight of 19.54 mg per 1,000 seeds which was 5.45% larger than wild-type seeds. To further test YDK1 role in regulating seed size, the inventors measured seed length and width of the ydk1 homozygous mutant and WT Col-0. The homozygous ydk1 produced seeds with an average length of 470 μm which was 3.75% larger than the WT length of 453 μm and the ydk1 produced seeds with an average width of 303 μm which was 13.1% wider than wild-type width of 268 μm. These quantitative results show that homozygous ydk1 mutants produced seeds that were statistically heavier, longer, and wider than WT but ydk1 seeds were still smaller than the arf8-2 seeds.

To examine whether the ydk1 mutation has a parent-of-origin effect on seed size, the inventors reciprocally crossed WT with the first generation of the ydk1 homozygous plants and measured the seed's weight, length, and width. Manual cross-pollinated WT×WT and ydk1×ydk1 were used as controls.

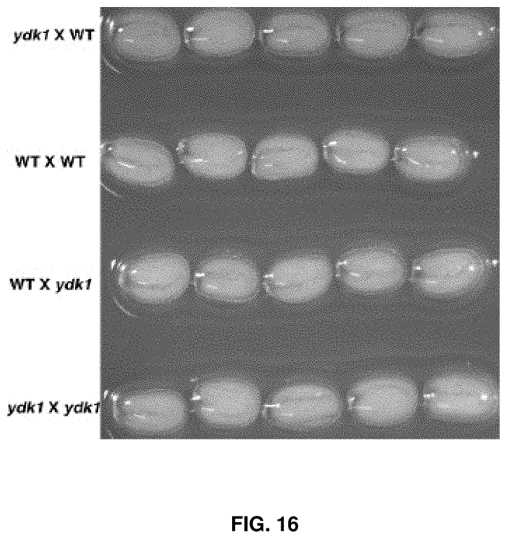

As shown in FIG. 16, the seed weight, length, and width were all larger for the homozygous ydk1 mutant as compared to WT Col-0 seeds. The ydk1 seeds were approximately 12% heavier, 4% longer and 9% wider than the wild-type (Table S4). The average weight of 1000 ydk1 seeds were 24.86 mg which was 12.00% larger than the WT of 22.20 mg. The average length of the ydk1 seeds was 534 μm which was 4.30% longer than the WT of 512 μm and the average width of the ydk1 mutant was 336 μm which is 9.45% wider than wild-type.

F1 seeds that were generated when the maternal parent was a homozygous ydk1 plant crossed with a paternal wild-type plant produced seeds that weight on average around 23.40 mg which was an increase of +5.41% as compared to the wild-type weight of 22.20 mg. The maternal ydk1 mutant (ydk1×WT) produced seeds which had weight, length, and width increased by 5.41%, 0.98%, and 8.79%, respectively, as compared to the WT cross (WT×WT). In the reciprocal cross (WT×ydk1), the F1 seeds had weight, length, and width increased by 11.10%, 2.54%, and 8.47%, respectively, when compared to the WT cross (WT×WT). The results from the genetic crosses showed that either the paternal ydk1 mutant or maternal ydk1 mutant was sufficient to produce heavier, longer, and wider seeds than wild-type. While the paternal ydk1 mutant seeds were heavier and longer than the maternal ydk1 mutant, they were slightly thinner but the differences between them were not statistically significant. Having both mutant ydk1 alleles though did produce the largest and longest seeds, but it seems like having either a paternal or maternal mutant allele was sufficient to produce heavier, longer, and wider seeds than wild-type.

Expression of ANT, AP2, and CYCD3 were altered in the arf8-2 and ydk1 seeds mutant seed. APETALA (AP2) a member of the AP2 DNA binding domain class of transcription factors has been shown to control seed size. AP2 has been suggested to act in the endosperm to influence seed size and has been shown to increase the number of cells in mature embryo (Ohto, Fischer et al., 2005). Morphological development of embryos is delayed in ap2 mutants. AINTEGUMENATA (ANT) has been shown to regulate cell division. Gain of ANT function has been shown to increase the cell number in Arabidopsis by regulating cell division in integuments during ovule development. Regulation of the cell cycle by ANT is mediated at least in part by extending the expression of CYCD3 (also shown as CYCD3;1 or CYCD3-1), a D-type cyclin that integrates cell cycle entry with exogenous signals such as hormones, including auxin. CYCD3 expression was active in the fertilized ovule and embryo signifying that it has an AP2 important role during embryo and endosperm development.

Since arf8-2 has been shown to increase the number of embryo and endosperm cells, the inventors wondered if arf8-2 will have increase expression of AP2, ANT, and CYCD3 since they have been showed in the previous research to regulate the development of those seed tissues and seed size. Total RNA was extracted from arf8-2, ydk1, and WT Col-0 seeds at two different time stages of 3 DAP and 7 DAP to see AP2, ANT, and CYCD3 expression in those seeds and then semi-quantitative RT-PCR was performed to analyze their expression.

Since the inventors saw that arf8-2 mutants had an increased in the number of embryo cells as compared to wild-type, they wondered if arf8-2 could have an increased expression of AP2 in developing seeds when compared to WT Col-0. They saw that expression was highest for arf8-2 at 3 DAP and 7 DAP. While ydk1 had similar expression of AP2 as WT; AP2 expression in arf8 was significantly higher than in WT at both 3 and 7 DAP (FIG. 4).

For ANT and CYCD3, the inventors saw that increased expression of those genes leads to increase seed size. Expression of ANT under the control of the constitutive 35S promoter results in the development of larger floral organs and stimulates cell growth. ANT and CYCD3 expression was low or undetectable at 3, but its expression in arf8-2 at 7DAP was significantly higher than in WT; ANT and CYCD3 expression in ydk1 was significantly higher than in WT at 7DAP yet ydk1 expression was still less than arf8-2 expression. These results show that loss of the repressive transcription factor ARF8 could derepress expression of ANT and CYCD3 which would lead to increase seed size. The higher expression of AP2 could lead to increase cell division and growth that occurs earlier in arf8-2 mutants and later on in ydk1 mutants explaining the larger size of the arf8-2 mutant in comparison with ydk1.

TABLE 3
Effects of the ydk1 mutation on seed size in Arabidopsis
(WT, wilde-type Col-0; ydk1, the homozygous ydk1).

| Genotype | Weight^{a} | Change^{b} | Length^{c} | Change^{b} | Width^{c} | Change^{b} |
|---|---|---|---|---|---|---|
| Wild Type | 18.53 ± 0.094 | — | 453 ± 3.30 | — | 268 ± 2.49 | — |
| ydk1 | 19.54 ± 0.102 | +5.45% | 470 ± 2.94 | +3.75%% | 303 ± 3.74 | +13.1% |

^{a}One thousand seeds were weighed. Weight (mg) per 1,000 seeds is shown. Standard deviation for thee replicates is shown.
^{b}Change is relative to wild-type seed.
^{c}Average length and width (μm) of 200 seeds is shown.

TABLE S1
Effects of the arf8-2 mutation, complementation, and overexpression on seed weight, length, and width

| Genotype | Weight^{a} | Change^{b} | Length^{c} | Change^{b} | Width^{c} | Change^{b} |
|---|---|---|---|---|---|---|
| Wild Type | 18.72 ± 0.112 | — | 448 ± 2.00 | — | 283 ± 5.51 | — |
| arf8-2 | 23.14 ± 0.118 | +23.6% | 506 ± 12.10 | +12.9% | 331 ± 9.29 | +17.0% |
| arf8-2 complement | 18.40 ± 0.173 | −1.71% | 438 ± 4.36 | −2.23% | 291 ± 7.81 | +2.82% |
| Wild Type | 17.96 ± 0.241 | −4.06% | 409 ± 6.81 | −8.71% | 265 ± 1.15 | −6.36% |
| overexpressed w/ARF8 | | | | | | |

^{a}One thousand seeds were weighed. Weight (mg) per 1,000 seeds is shown. Standard deviation for three replicates is shown.
^{b}Change is relative to wild-type seed.
^{c}Average length and width (μm) of 200 seeds is shown.

TABLE S2
Parent-of-origin effects of the arf8-2 mutation on F1 seed weight, length and width

| Genetic Cross | | | | | | | |
|---|---|---|---|---|---|---|---|
| Matemal | Paternal | Weight^{a} | Change^{b} | Length^{c} | Change^{b} | Width^{c} | Change^{b} |
| Wild Type | arf8-2 | 25.36 ± 0.12 | +16.0% | 544 ± 7.79 | +7.94% | 332 ± 8.60 | +8.35% |
| Wild Type | Wild Type | 21.86 ± 0.47 | — | 504 ± 13.3 | — | 305 ± 4.90 | — |
| arf8-2 | Wild Type | 26.58 ± 0.23 | +21.6% | 548 ± 4.11 | +8.73% | 329 ± 6.13 | +7.87% |
| arf8-2 | arf8-2 | 28.24 ± 0.41 | +29.2% | 563 ± 3.91 | +11.7% | 331 ± 3.09 | +8.52% |

^{a}One thousand seeds were weighed. Weight (mg) per 1000 seeds is shown. Standard deviation for three replicates is shown.
^{b}Change is relative to wild-type F₁ seed.
^{c}Average length and width (μm) of 200 seeds is shown.

TABLE S3
Parent-of-origin effects of the arf8-2 mutation on F1 cell numbers of embryo and endosperm

| Genetic Cross | | | | | |
|---|---|---|---|---|---|
| Maternal | Paternal | Cell number of embryo ^{a} | Change^{b} | Cell number of endosperm ^{a} | Change^{b} |
| Wild Type | arf8-2 | 425,832 ± 54,324 | +106% | 64,565 ± 28,826 | +53.7% |
| Wild Type | Wild Type | 206,277 ± 49,172 | — | 42,017 ± 4070 | — |
| arf8-2 | Wild Type | 398,819 ± 134,000 | +93.3% | 58,684 ± 12,340 | +38.2% |
| arf8-2 | arf8-2 | 503,580 ± 104,048 | +144% | 70,279 ± 25,995 | +67.3% |

^{a} Two hundred seeds were sorted. Standard deviation for three replicates is shown. Total sorted cell number of embryo of endosperm of 200 seeds is shown.
^{b}Change is relative to wild-type cross.

B. Discussion

Early experiments on ARF8 was centered on arf6-2 arf8-3 double mutant. While arf8 single mutant have delayed stamen development and shorter inflorescence stems, arf6-2 and arf8-3 double mutant caused infertility in both Arabidopsis and tomatoes (Nagpal et al., 2005, Liu et al., 2013). Goetz et al., 2006 showed that ARF8 was a negative regulator of fruit initiation. These results showed that ARF8 has a critical role in development. This study was the first to show that ARF8 has a critical role in not only development but seed development and seed size. arf8-2 homozygous mutants generated seeds that were approximately 24% heavier, 13% longer and 17% wider than WT Col-0 seeds. To understand the extent of the arf8 mutation on seed size, the inventors studied seed development by DIC to view embryo growth as well as seed size as the seed developed after pollination. the inventors found no abnormal growth but arf8-2 seeds did have larger cavity than WT Col-0 seeds especially after 5 DAP. This increase in seed cavity could be caused by an increase in embryo and endosperm size that makes up the interior portion of the seed.

Seed has three major components: the embryo which grows into a new plant, the endosperm which is a nutritive tissue nourishing the growing embryo and the seed coat which is maternally derived tissues and encloses the embryo sac. Embryo and endosperm coordinate their development with the surrounding seed coat to produce a seed. Since these tissues make up the bulk of the seed, the inventors wanted to see how arf8-2 mutation would affect embryo and endosperm growth. Fertilization of the egg cell by a sperm cell gives rise to the diploid embryo, while fertilization of the diploid central cell by the second sperm cell gives rise to the triploid endosperm. Exploiting differences in ploidy between these tissues allows us to separate the endosperm and embryo with nuclei staining and cell sorting.

Mechanical separation of arf8-2 seeds allowed us to separate endosperm and embryo tissues. By using FACS, the inventors were able to isolate embryo and endosperm nuclei based on their ploidy and count the number of embryo and endosperm nuclei/cells in the arf8-2 mutant and WT seeds. Using this method, the inventors observed that the arf8-2 mutation causes an increase number of embryo and endosperm cells compared to WT Col-0 at 9 DAP. The embryo was particularly affected as the arf8-2 mutation caused 154% increase in embryo cell number as compared to WT. The arf8-2 mutation caused 46% increase in endosperm cell number as compared to WT. These large changes in embryo and endosperm cell number were also seen in the F1 seed of the reciprocal cross between arf8-2 mutant and WT. The large increase in the cell number of endosperm and embryo tissue of arf8-2 seeds could explain the larger seed size of the arf8-2 mutants compared to WT seeds. Increase cell number of embryo and endosperm tissue could increase the amount of cell volume taken up by the embryo and endosperm thereby causing larger seed size than WT seeds.

Since the inventors saw an increase in seed size from arf8-2, they wondered if they could find any downstream target of arf8-2 that could also cause an increase in seed size. the inventors looked at the GH3 genes which are known as the auxin response gene. One of the genes the inventors found was YDK1. ARF8 is a known positive regulator of the YDK1 gene and they wondered if knocking this gene out would cause a similar phenotype as ARF8. They saw that knocking out YDK1 cause a similar phenotype. The ydk1 mutant seed was 5% larger seed than WT. While this difference is not as large as arf8-2, ydk1 mutants actually produced as much seeds as WT Col-0 or more seed per silique than WT Col-0 which is different than arf8-2 mutants which produced less seeds per silique than WT. Since the increase in ydk1 seeds were not as large as arf8-2 seeds, it is possible that a transcription factor like ARF8 could affect multiple genes that affect seed size.

After seeing the large increases in cell number, the inventors wondered if arf8-2 could promote cell division, give rise to more cells and thus cause larger seed size. Therefore, they tested AP2, ANT, and CYCD3 expression in arf8-2, ydk1, and WT seeds. They saw that expression of AP2, ANT, and CYCD3 increased in the arf8-2 seed as compared to WT seeds at 7DAP; AP2, ANT, and CYCD3 expression increased in the ydk1 seed at both 7 DAP and 21 DAP as compared to WT seeds.

ant-1 mutants had tissues that were smaller and had fewer cells, while 35S::ANT (an overexpression ANT transgene) was shown to extend the cell cycle by increasing CYCD3 expression thereby sustaining cell proliferation for longer and increasing the number of cell division which could lead to an increase in seed size (Mizukami and Fischer 1999).

It is likely that the arf8-2 mutation induces expression of ANT, a transcription factor involved in organ growth and cell division control and CYCD3, a D-type cyclin involved in cell cycle entry expression along with AP2, which is involved in regulating embryo cell number and size, thus resulting in larger seeds in the arf8 mutant. This is similar to the results found by Varaud et al., 2011 that proposed that ARF8 is a negative regulator of petal growth. They found that petal overgrowth and a larger petal phenotype in arf8-3 was due to an increased in cell production leading to an increase in cell number. They found that ARF8 had a binding partner called BIGPETALp (BPEp) a basic helix-loop-helix (bHLH) transcription factor that was preferentially expressed in petals.

When auxin is absent, AUXIN/INDOLE-3-ACETIC ACID (Aux/IAA) repressor proteins inhibit the transcriptional regulatory function of (ARFs). High auxin concentration promotes interaction of auxin and Aux/IAAs with F-box proteins of the TRANSPORT INHIBITOR RESPONSE 1 (TIR1)/AUXIN-BINDING F-BOX (AFB) family. These proteins are part of a ubiquitin ligase complex that ubiquitinates Aux/IAAs and thereby targets them for degradation in the 26S proteasome. This revokes repression of ARFs, which can in turn regulate auxin-responsive genes.

It was proposed that there are 5 ARFs that functions as transcriptional activators when tested on auxin response genes (Tiwari et al., 2003; Ulmasov et al., 1999), yet ARF8 has been described as a negative regulator of fruit initiation and petal growth (Goetz et al., 2006; Varaud et al., 2011). One way to explain this contradiction is YDK1, which could function as a negative component in auxin signaling. YDK1 transgenic plants show a dwarf phenotype with short primary root and short hypocotyl (Takase et al., 2004). ARF8 a transcriptional activator could activate YDK1 which could be a negative regulator of cell division. Mutant arf8-2 would not activate YDK1 and leads to larger seed size. While this proposed mechanism is possible ydk1 mutant did not produce as large of seeds as arf8-2, ARF8 could regulate other unknown downstream genes. The timing of increase cell division and expression of other relevant genes to increase cell division remains unknown. Also, the direct targets of ARF8 and its partners needed to be studied to clarify the mechanism of ARF8 function.

ARF2 was shown to have an effect on seed size. Mutant arf2-8 caused larger seed size than WT seeds. Their experiments further showed that ARF2 had a parent-of-origin effect on seed size. Maternal arf2-8 mutant crossed with paternal WT produced much larger seeds than maternal WT crossed with paternal arf2-8 mutant (Schruff et al., 2005). Reciprocal crosses on arf8-2 and WT to see if there was any parent-of-origin effect on seed size. The results were different than ARF2. Using reciprocal crosses to determine the genetic basis of this phenotype, arf8-2 mutants cause larger seed size whether it was a maternal or paternal mutant. Homozygous arf8-2 mutants produced the largest seeds that were on average 29.2% larger than WT, while paternal amd maternal arf8-2 mutants produced seeds that were 16.0% and 21.6% larger than WT seeds.

arf8-2 might be a dominant mutation. Losing this gene could cause larger seed size regardless of parent-of-origin. Because there are 22 full length Arabidopsis ARF genes that encode proteins with full-length DNA-binding domain (DBD) and one partial-length gene (ARF23) with a stop codon in its DBD that can recognize and compete for TGTCTC target sites in promoters of auxin response genes (Guilfoyle and Hagen 2001, Tiwari et al., 2003; Ulmasov et al., 1999), loss of some ARF8 could allow for other ARFs to bind target genes instead of activating or repressing different pathway than in WT seeds.

After finding a downstream target of ARF8-YDK1 that could affect seed size, the inventors wanted to examine if an upstream gene that regulates ARF8 could also affect seed size. miRNA167 which has been shown to regulate ARF8 has maternal control of seed development. While maternal miRNA167 crossed with paternal WT crosses failed to produce normal endosperm and embryo, the inventors did not see this maternal effect on seed size. miRNA167a knockout produced seeds that were similar in size to WT.

While this study had shown that ARF8 has a profound effect on seed size, many questions still remains. A downstream mechanism involving YDK1 and activating other transcription factors to increase cell division and cell number was shown but other proteins or binding partners of ARF8 or YDK1 in this pathway are unknown. Larger seed size was explained by increase number of cells but change in seed size can be explained by either an alteration in the size of each individual cell or the number of cells. Examining the size of each cell would help explain this process more fully. Lastly, methylation has been shown to have an effect on seed size and ARF8 was found in the screening from a MET1 microarray. Looking at the methylation of the ARF8 locus and ARF8 gene expression in met1 mutant plants could provide the upstream regulation of ARF8 and explain the link between methylation, auxin, and seed development.

### Example 3—Results and Discussion

A. Results

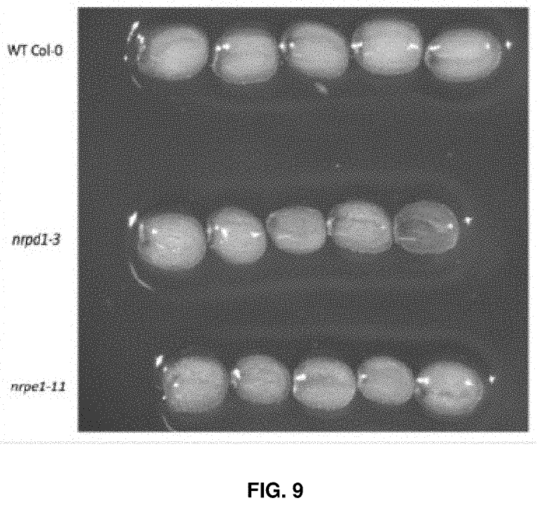

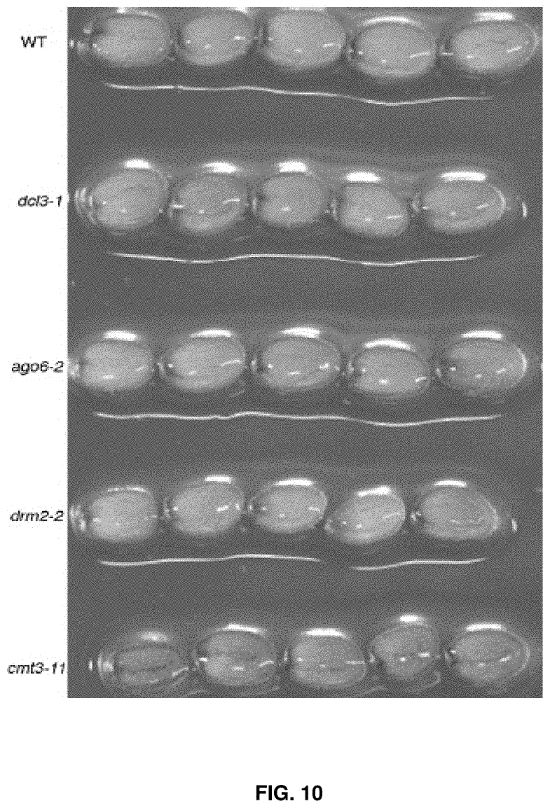

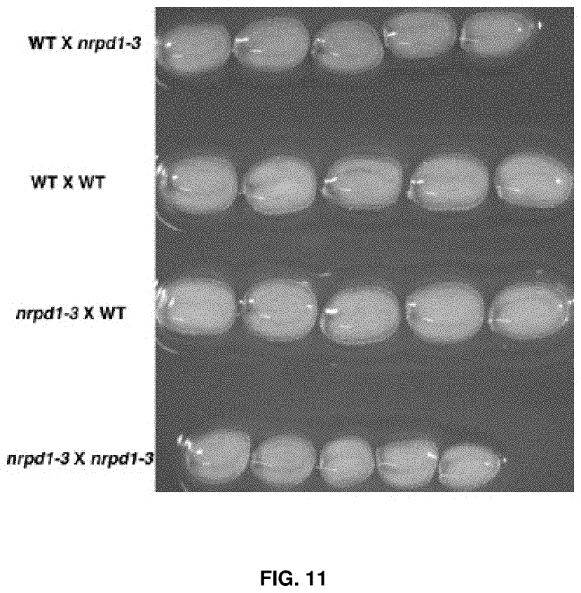

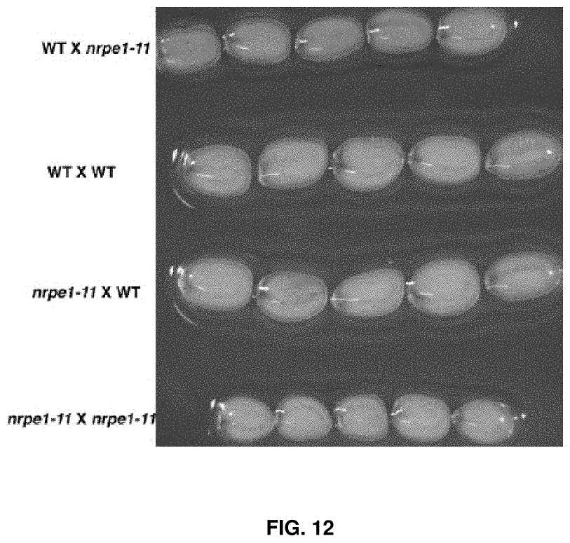

Mutations to RNA Polymerases of the Canonical RdDM Affect Seed Size. The inventors examined if knocking out key components to the RdDM pathway which affects CHH methylation would have an effect on seed size. First, they started with RNA Polymerase IV largest subunit NRPD and RNA Polymerase V largest subunit NRPE. As shown in FIG. 9, the nrpd1-3 homozygous mutant seed was smaller than wild-type. To assess quantitatively the effect of NRPD1 in regulating seed size, the inventors measured seed weight of the nrpd1-3 mutant and WT. As shown in Table 4, the nrpd1-3 mutant on average weighed around 17.03 mg per 1000 seeds while wild-type weighed around 18.36 mg. This represents 7.24% decrease in weight. To further test NPRD1 role in regulating seed size, the inventors measured seed length and width. As shown in Table 4, the average wild-type seed length was approximately 454 μm while the nrpd1-3 mutant had an average seed length of 416 μm, representing 8.37% decrease. They inventors saw a similar decrease in seed width with nrpd1-3 width on average 253 μm which was a 6.30% decrease as compared to the wild-type width of 283 μm.

To assess quantitatively the effects of NRPE1 in regulating seed size, the inventors measured seed weight of the nrpe1-11 mutant and WT Col-0. As shown in Table 4, the mutant nrpe1-11 produced significantly smaller seeds than WT. The nrpd1-3 mutant weighed around 16.97 mg per 1000 seeds while wild-type weighed around 18.36 mg, representing a 7.57%. As shown in Table 4, the mutant nrpe1-11 had seed length of 410 μm and width of 253 μm, representing 9.69% and 6.30% decrease, respectively as compared to WT.

Mutations to Other Components of the RdDM Pathway Affects Seed Size. After examining how knocking out RNA Polymerase IV and V of the RdDM pathway causes smaller seed size than WT, the inventors examined if knocking out other key components to the RdDM pathway would have an effect on seed size. They saw that each mutant produced lighter seeds than WT. As shown in Table 5, the dcl3-1 mutant on average weighed around 18.04 mg per 1000 seeds while wild-type weighed around 18.61 mg, representing 3.06%. decrease. The average wild-type seed length was approximately 468 μm while dcl3-1 mutants had an average seed length of 465 μm. represents 0.64% decrease. The dcl3-1 mutant has seed width of 273 μm which was 3.53% decrease as compared to the WT width of 283 μm.

As shown in Table 5, the ago6-2 mutant seed weighed around 18.15 mg per 1000 seeds while wild-type weight around 18.61 mg, representing 2.47% decrease. The average wild-type seed length was approximately 468 μm, similar to the ago6-2 mutant of 469 μm. The ago6-2 had a seed width of 271 μm, representing 4.24% decrease as compared to the wild-type width of 283 μm. Lighter F1 seed was generated with the drm2-2 homozygous mutant as compared to wild-type. The drm2-2 mutant had seed weight, length and width of 17.41 mg, 449 μm, and 258 μm, representing 6.45%, 4.06%, and 8.83% decrease than WT, respectively (Table 5). Lighter F1 seed was generated with the cmt3-11 homozygous mutant as compared to wild-type. The cmt3-11 mutant had seed weight, length and width of 17.63 mg, 441 μm, and 276 μm, representing 5.27%, 5.77%, and 2.47% decrease than WT, respectively (Table 5).

While dcl3-1, ago6-2, drm2-2, and cmt3-11 did not produce significantly smaller seeds than WT as nrpd1-3 and nrpe1-11, all mutants produced smaller seeds than WT; drm2-2 and cmt3-11 produced significantly smaller seeds than dcl3-1 and ago6-2. These results show that the RdDM pathway has an effect on seed size and that loss of CHH methylation produces smaller seeds than WT Col-0.

nrpd1-3 and nrpe1-11 Have Potential Parent-of-Origin Effects on Seed Size. To assess the effect of maternal versus paternal genome hypomethylation on seed size, the inventors reciprocally crossed wild-type with the first generation of the nrpd1-3 homozygous plants. In the cross of WT×nrpd1-3, the F1 seed weighed 20.86 mg per 1000 seeds, 487 μm in length, and 292 μm in width which were 7.09% lighter, −6.68% shorter, and 5.19% thinner than WT Col-0, respectively. So smaller seeds were produced but not as small as in homozygous nrpd1-3 mutant (Table 6). In the cross of nrpd1-3×WT, the F1 seed weighed 22.34 mg per 1000 seeds, 492 μm in length, and 325 μm in width which were 0% change, 5.93% decrease, and 5.52% increase as compared to WT, respectively. So smaller seeds were produced but not as small as in homozygous nrpd1-3 mutant (Table 6).

To assess the NRPE1 parent-of-origin effect on seed size, the inventors reciprocally crossed wild-type with the first generation of the nrpe1-11 homozygous plants. In the cross of WT×nrpe1-11, the F1 seed weighed 21.52 mg per 1000 seeds, 488 μm in length, and 298 μm in width which were 2.45% lighter, 4.50% shorter, and 2.93% thinner than WT Col-0, respectively. So smaller seeds were produced but not as small as in the homozygous nrpd1-3 mutant. In the cross of nrpe1-11×WT, the F1 seed weighed 22.66 mg per 1000 seeds, 523 μm in length, and 326 μm in width which were 2.72%, 2.35% shorter, and 0.33% increase as compared to WT, respectively. So smaller seeds were produced but not as small as in the homozygous nrpd1-3 mutant.

TABLE 4
Effects of Mutations to RNA Polymerase IV and V on Seed Weight, Length and Width

| Genotype | Weight^{a} | Change^{b} | Length^{c} | Change^{b} | Width^{c} | Change^{b} |
|---|---|---|---|---|---|---|
| Wid Type | 18.36 ± 0.122 | — | 454 ± 4.50 | — | 270 ± 3.74 | — |
| nrpd1-3 | 17.03* ± 0.040 | −7.24%* | 416* ± 3.00 | −8.37%* | 253* ± 5.29 | −6.30%* |
| nrpe1-11 | 16.97* ± 0.066 | −7.57%* | 410* ± 11.53 | −9.69%* | 253* ± 3.51 | −6.30%* |

^{a}One thousand seeds were weighed. Weight per 1,000 seeds is shown. Standard deviation for three replicates is shown.
^{b}Change is relative to wild-type seed.
^{c}Average length and width of 200 seeds is shown.
*Indicates a statistical difference based on unpaired t-test.

TABLE 5
Effects of Mutations to Other Components of the
RdDM Pathway on Seed Weight, Length and Width

| Genotype | Weight^{a} | Change^{b} | Length^{c} | Change^{b} | Width^{c} | Change^{b} |
|---|---|---|---|---|---|---|
| Wild Type | 18.61 ± 0.134 | — | 468 ± 7.41 | — | 283 ± 3.09 | — |
| dcl3-1 | 18.04 ± 0.212 | −3.06% | 465 ± 8.18 | −0.64% | 273 ± 2.62 | −3.53%* |
| ago6-2 | 18.15 ± 0.212 | −2.47% | 469 ± 10.40 | +0.21% | 271 ± 2.16 | −4.24%* |
| drm2-2 | 17.41* ± 0.035 | −6.45%* | 449 ± 7.54 | −4.06%* | 258 ± 0.94 | −8.83%* |
| cmt3-11 | 17.63* ± 0.240 | −5.27%* | 441 ± 10.80 | −5.77%* | 276 ± 5.35 | −2.47% |

^{a}One thousand seeds were weighed. Weight per 1,000 seeds is shown. Standard deviation for three replicates is shown.
^{b}Change is relative to wild-type seed.
^{c}Average length and width of 200 seeds is shown.
*Indicates a statistical difference based on unpaired t-test.

TABLE 6
Parent-of-Origin Effects of the nrpd-1-3 Mutation on F1 Seed Weight, Length and Width

| Genetic Cross | | | | | | | |
|---|---|---|---|---|---|---|---|
| Maternal | Paternal | Weight^{a} | Change^{b} | Length^{c} | Change^{b} | Width^{c} | Change^{b} |
| Wild Type | nrpd1-3 | 20.86* ± 0.404 | −7.09%* | 487* ± 4.55 | −6.88%* | 292* ± 1.89 | −5.19%* |
| Wild Type | Wild Type | 22.34 ± 0.058 | — | 523 ± 3.30 | — | 308 ± 2.45 | — |
| nrpd1-3 | Wild Type | 22.34 ± 0.090 | 0% | 492* ± 5.25 | −5.939%* | 325* ± 0.47 | +5.52% |
| nrpd1-3 | nrpd1-3 | 18.60* ± 0.436 | −16.7%* | 471* ± 8.98 | −9.04%* | 298* ± 3.27 | −3.25%* |

^{a}One thousand seeds were weighed. Weight per 1,000 seeds is shown. Standard deviation for three replicates is shown.
^{b}Change is relative to wild-type F₁ seed.
^{c}Average length and width of 200 seeds is shown.
*Indicates a statistical difference based on unpaired t-test.

B. Discussion

RdDM specific RNA Polymerase IV and V (nrpd1-3 and nrpe1-11) as well as drm2-2 and cmt3-11 homozygous mutations decrease seed size. DNA methylation is essential for regulating development in plants (Martienssen and Colot, 2001; Bender 2004; Gehring et al., 2004). Both MET1 and DECREASE IN DNA METHYLATION1 (DDM1) is required for normal pattern of genomic DNA methylation. Previous research by Xiao et al., 2006 showed that genome hypomethylation at CG sites by mutations to MET1 or DDM1 affect seed size. Could loss of CHG or CHH methylation also affect seed size? By mutating the largest subunit RNA Polymerase IV (nrpd1-3 mutant) and RNA Polymerase V (nrpe1-11) of the RdDM pathway smaller seeds were produced. Both nrpd1-3 and nrpe1-11 mutation produce seeds that were around 7% lighter, 8% shorter, and 6% thinner than wild-type.

After finding these results, the inventors wanted to see if any other components of the RdDM pathway could produce smaller seed size like nrpd1-3 and nrpe1-11. Examining five mutants important to the RdDM pathway showed that even though all mutants produced smaller seeds than WT, only drm2-2 and cmt3-11 produced similar phenotype as nrpd1-3 and nrpe1-11. Components such as dcl3-1 and ago6-2 can be explored in more details since there are multiple DICER-LIKE proteins such as DCL2, DCL3, and DCL4. Likewise, there are multiple ARGONAUTE proteins AGO1, AGO4, and AGO6. Triple homozygous mutants knocking out each component could provide evidence that ARGONAUTE and DICER-LIKE proteins could also produce smaller seed size than WT.

This research has shown that the canonical RdDM pathway has an effect on seed size. Exploring the non-canonical RdDM pathway and its effect on seed size will be necessary to see how de novo CHH methylation affects seed development. Xiao et al. (2006) showed that MET1 and DDM1 affected seed size and now the RdDM pathway is shown to affect seed size therefore DNA methylation likely regulates genes and pathway affecting seed size and finding targets of

DNA methylation can reveal the connection between DNA methylation and seed size. DNA methylation is very important for seed development. Seeds also undergo dynamic DNA methylation changes throughout their development. CHH methylation was found changing the most compared with CG and CHG methylation during soybean seed development, and 2136 genes regulated by CHH methylation were found (An et al., 2017). The inventors found that the largest subunits of RNA Polymerase IV and V involved in the RdDM pathway and affect CHH methylation can significantly affect seed size, future efforts will involve identifying Arabidopsis genes that are regulated by CHH methylation. This will provide a better understanding how RdDM can affect seed development. This may also show that CHH methylation can affect target genes showing a potential pathway on how methylation can affect seed development and size.

Previous research mentioned a reported tradeoff between seed number and size in many species (Harper et al., 1970), including Arabidopsis (Alonso-Blanco et al., 1999; Meyer et al., 2004). This led me to investigate this further by examining the amount of seeds produced by nrpd1-3 and nrpe1-11 mutants per silique and compare that the WT Col-0. The nrpd1-3 and nrpe1-11 had little effect on seed set since they roughly produced the same amount of seeds as WT Col-0. This shows that smaller seed size was not due to an increase in seed number.

RdDM specific RNA Polymerase IV and V (nrpd1-3 and nrpe1-11) have a parent-of-origin effect on seed size. Genetic crosses with the nrpd1-3 and nrpe1-11 mutants were also analyzed for their influence on seed size. While the parent-of-origin effect wasn't as drastic as in the met1-6 mutant, the nrpd1-3 and nrpe1-11 still produced seeds with a slight parent-of-origin effect. The F1 seed with paternal nrpd1-3 and nrpe1-11 mutants produced smaller seeds than WT which is consistent with paternal excess crosses and the met1-6 mutant. The F1 seed with maternal nrpd1-3 and nrpe1-11 was as large or larger than WT which is consistent with maternal excess crosses and the met1-6 mutant. Genetic crosses of the other RdDM mutants should be performed to further understand if the other components in the pathway has a parent-of-origin of seed development.

How does hypomethylation of maternal and paternal genomes influence seed size? Genomic imprinting in the endosperm has been shown to regulate seed size and development in plants (Haig and Westoby, 1991; Kohler, Page et al., 2005; Gehring, Huh et al., 2006; Jullien and Berger, 2006); One theory to explain the evolution of genomic imprinting is the parental conflict theory (Haig and Westoby, 1991). The theory predicts that alleles of some genes active in the offspring in allocating resources from maternal tissue to offspring will have different expression patterns depending on the parent of origin. For endosperm activators, the maternal allele is likely silenced to conserve resources and the paternal allele is expressed so that maternal parent expends resources for each individual of her offspring.

Thus, the maternal alleles of endosperm activators, which are normally silenced, are expressed and lead to larger F1 seeds. In the cross of a wild-type female with the nrpd1-3 and nrpe1-11 male parent, the maternal-derived, wild-type genome is not modified and the paternal-derived genome is hypomethylated. Thus, normally silenced paternal alleles of endosperm repressors are expressed, which leads to repressed endosperm development and smaller seeds. Therefore, DNA methylation likely regulates genes and pathways affecting seed size. The target genes and pathways are not known. What makes this field particularly challenging is that certain genes can be upregulated and downregulated within the same plant depending only on parent-of-origin. This complex interplay between parent-of-origin and seed size could reveal more insights into genomic imprinting, seed development, and plant evolutionary history that scientists have been unable to elucidate.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Adams, S., R. Vinkenoog, et al., (2000). “Parent-of-origin effects on seed development in Arabidopsis thaliana require DNA methylation.” Development 127: 2493-2502.
An, Y. C., W. Goettel, et al., (2017). “Dynamic Changes of Genome-Wide DNA Methylation during Soybean Seed Development.” Sci Rep 7(1): 12263.
Bartee, L., F. Malagnac, et al., (2001). “Arabidopsis cmt3 chromomethylase mutations block non-CG methylation and silencing of an endogenous gene.” Genes Dev. 15: 1753-1758.
Bird, A. (2002). “DNA methylation patterns and epigenetic memory.” Genes Dev 16(1): 6-21.
Borges, F., J. S. Parent, et al., (2018). “Transposon-derived small RNAs triggered by miR845 mediate genome dosage response in Arabidopsis.” Nat Genet 50(2): 186-192.
Butenko, Y. and N. Ohad (2011). “Polycomb-group mediated epigenetic mechanisms through plant evolution.” Biochim Biophys Acta 1809(8): 395-406.
Cao, X. and S. E. Jacobsen (2002). “Locus-specific control of asymmetric and CpNpG methylation by the DRM and CMT3 methyltransferase genes.” Proc. Natl. Acad. Sci. USA 99: 16491-16498.
Choi, Y., M. Gehring, et al., (2002). “DEMETER, a DNA Glycosylase Domain Protein, Is Required for Endosperm Gene Imprinting and Seed Viability in Arabidopsis.” Cell 110: 33-42.
Crevillen, P., H. Yang, et al., (2014). “Epigenetic reprogramming that prevents transgenerational inheritance of the vernalized state.” Nature 515(7528): 587-90.
Erdmann, R. M., P. R. V. Satyaki, et al., (2017). “A Small RNA Pathway Mediates Allelic Dosage in Endosperm.” Cell Rep 21(12): 3364-3372.
Finnegan, E. J. and E. S. Dennis (1993). “Isolation and identification by sequence homology of a putative cytosine methyltransferase from Arabidopsis thaliana.” Nucl. Acids Res. 21: 2383-2388.
Finnegan, E. J. and K. A. Kovac (2000). “Plant DNA Methyltransferases.” Plant Mol Biol 43: 189-201.
Finnegan, E. J., W. J. Peacock, et al., (1996). “Reduced DNA methylation in Arabidopsis results in abnormal plant development.” Proc. Natl. Acad. Sci. USA 93: 8449-8454.
Finnegan, E. J., W. J. Peacock, et al., (2000). “DNA methylation, a key regulator of plant development and other processes.” Curr Opin Genet Dev 10(2): 217-23.
Fujimoto, R., T. Sasaki, et al., (2006). “Characterization of DNA methyltransferase genes in Brassica rapa.” Genes Genet Syst 81(4): 235-42.
Gong, Z., T. Morales-Ruiz, et al., (2002). “ROS1, a Repressor of Transcriptional Gene Silencing in Arabidopsis, Encodes a DNA Glycosylase/Lyase.” Cell 111: 803-814.
Huang, Y., L. Ji, et al., (2009). “Structural insights into mechanisms of the small RNA methyltransferase HEN1.” Nature 461(7265): 823-7.
Ibarra, C. A., X. Feng, et al., (2012). “Active DNA demethylation in plant companion cells reinforces transposon methylation in gametes.” Science 337(6100): 1360-4.
Jacobsen, S. E., H. Sakai, et al., (2000). “Ectopic hypermethylation of flower-specific genes in Arabidopsis.” Curr. Biol. 10: 179-186.
Ji, L. and X. Chen (2012). “Regulation of small RNA stability: methylation and beyond.” Cell Res 22(4): 624-36.
Johnson, C., A. Kasprzewska, et al., (2009). “Clusters and superclusters of phased small RNAs in the developing inflorescence of rice.” Genome Res 19(8): 1429-40.
Jullien, P. E., A. Katz, et al., (2006). “Polycomb group complexes self-regulate imprinting of the Polycomb group gene MEDEA in Arabidopsis.” Curr Biol 16(5): 486-92.
Kakutani, T., J. A. Jeddeloh, et al., (1996). “Developmental abnormalities and epimutations associated with DNA hypomethylation mutations.” Proc. Natl. Acad. Sci. USA 93: 12406-12411.
Kankel, M. W., D. E. Ramsey, et al., (2003). “Arabidopsis MET1 Cytosine Methyltransferase Mutants.” Genetics 163: 1109-1122.
Kawakatsu, T., J. R. Nery, et al., (2017). “Dynamic DNA methylation reconfiguration during seed development and germination.” Genome Biol 18(1): 171.
Kim, M., H. Ohr, et al., (2008). “Temporal and spatial downregulation of Arabidopsis MET1 activity results in global DNA hypomethylation and developmental defects.” Mol Cells 26(6): 611-5.
Komiya, R., H. Ohyanagi, et al., (2014). “Rice germline-specific Argonaute MEL1 protein binds to phasiRNAs generated from more than 700 lincRNAs.” Plant J 78(3): 385-97.
Lanfear, R. (2018). “Do plants have a segregated germline?” PLoS Biol 16(5): e2005439.
Lee, H., R. L. Fischer, et al., (2003). “Arabidopsis LEAFY COTYLEDON1 represents a functionally specialized subunit of the CCAAT binding transcription factor.” Proc Natl Acad Sci USA 100(4): 2152-6.
Lei, M., H. Zhang, et al., (2015). “Regulatory link between DNA methylation and active demethylation in Arabidopsis.” Proc Natl Acad Sci USA 112(11): 3553-7.
Li, E. (2002). “Chromatin Modification and Epigenetic Reprogramming in Mammalian Development.” Nat. Rev. Genet. 3: 662-673.
Li, E., T. H. Bestor, et al., (1992). “Targeted mutation of the DNA methyltransferase gene results in embryonic lethality.” Cell 69(6): 915-26.
Li, Q., S. R. Eichten, et al., (2014). “Genetic perturbation of the maize methylome.” Plant Cell 26(12): 4602-16.
Lin, J. Y., B. H. Le, et al., (2017). “Similarity between soybean and Arabidopsis seed methylomes and loss of non-CG methylation does not affect seed development.” Proc Natl Acad Sci USA 114(45): E9730-E9739.
Lindroth, A. M., X. Cao, et al., (2001). “Requirement of CHROMOMETHYLASE3 for maintenance of CpXpG methylation.” Science 292: 2077-2080.
Lira-Medeiros, C. F., C. Parisod, et al., (2010). “Epigenetic variation in mangrove plants occurring in contrasting natural environment.” PLoS One 5(4): e10326.
Lotan, L., M.-A. Ohto, et al., (1998). “Arabidopsis LEAFY COTYLEDON1 is sufficient to induce embryo development in vegetative cells.” Cell 93: 1195-1205.
Lu, J., C. Zhang, et al., (2012). “Maternal siRNAs as regulators of parental genome imbalance and gene expression in endosperm of Arabidopsis seeds.” Proc Natl Acad Sci USA 109(14): 5529-34.
Luna, E. and J. Ton (2012). “The epigenetic machinery controlling transgenerational systemic acquired resistance.” Plant Signal Behav 7(6): 615-8.
Luo, M., P. Bilodeau, et al., (2000). “Expression and parent-of-origin effects for FIS2, MEA, and FIE in the endosperm and embryo of developing Arabidopsis seeds.” Proceedings of the National Academy of Sciences of the United States of America 97(19): 10637-10642.
Mallory, A. C., T. Elmayan, et al., (2008). “MicroRNA maturation and action—the expanding roles of ARGONAUTEs.” Curr Opin Plant Biol 11(5): 560-6.
Manning, K., M. Tor, et al., (2006). “A naturally occurring epigenetic mutation in a gene encoding an SBP-box transcription factor inhibits tomato fruit ripening.” Nat Genet 38(8): 948-52.
Martienssen, R. A. and V. Colot (2001). “DNA methylation and epigenetic inheritance in plants and filamentous fungi.” Science 293: 1070-1074.
Martinez, G., P. Wolff, et al., (2018). “Paternal easiRNAs regulate parental genome dosage in Arabidopsis.” Nat Genet 50(2): 193-198.
Martinez, G., P. Wolff, et al., (2018). “Paternal easiRNAs regulate parental genome dosage in Arabidopsis.” Nature Genetics 50(2): 193-198.
Mirouze, M., J. Reinders, et al., (2009). “Selective epigenetic control of retrotransposition in Arabidopsis.” Nature 461(7262): 427-30.
Miura, A., S. Yonebayashi, et al., (2001). “Mobilization of transposons by a mutation abolishing full DNA methylation in Arabidopsis.” Nature 411: 212-214.
Mosher, R. A., C. W. Melnyk, et al., (2009). “Uniparental expression of PolIV-dependent siRNAs in developing endosperm of Arabidopsis.” Nature 460(7252): 283-6.
Narsai, R., Q. Gouil, et al., (2017). “Extensive transcriptomic and epigenomic remodelling occurs during Arabidopsis thaliana germination.” Genome Biol 18(1): 172.
Nonomura, K., A. Morohoshi, et al., (2007). “A germ cell specific gene of the ARGONAUTE family is essential for the progression of premeiotic mitosis and meiosis during sporogenesis in rice.” Plant Cell 19(8): 2583-94.
Oliver, C., J. L. Santos, et al., (2016). “Accurate Chromosome Segregation at First Meiotic Division Requires AGO4, a Protein Involved in RNA-Dependent DNA Methylation in Arabidopsis thaliana.” Genetics 204(2): 543-553.
Onodera, Y., J. R. Haag, et al., (2005). “Plant nuclear RNA polymerase IV mediates siRNA and DNA methylation-dependent heterochromatin formation.” Cell 120(5): 613-22.
Papa, C. M., N. M. Springer, et al., (2001). “Maize chromomethylase Zea methyltransferase2 is required for CpNpG methylation.” Plant Cell 13(8): 1919-28.
Pavlopoulou, A. and S. Kossida (2007). “Plant cytosine-5 DNA methyltransferases: structure, function, and molecular evolution.” Genomics 90(4): 530-41.
Pelletier, J. M., R. W. Kwong, et al., (2017). “LEC1 sequentially regulates the transcription of genes involved in diverse developmental processes during seed development.” Proc Natl Acad Sci USA 114(32): E6710-E6719.
Pignatta, D., R. M. Erdmann, et al., (2014). “Natural epigenetic polymorphisms lead to intraspecific variation in Arabidopsis gene imprinting.” Elife 3: e03198.
Pradhan, S., M. Cummings, et al., (1998). “Isolation, characterization and baculovirus-mediated expression of the cDNA encoding cytosine DNA methyltransferase from Pisum sativum.” Nucleic Acids Res 26(5): 1214-22.
Rea, M., W. Zheng, et al., (2012). “Histone H1 affects gene imprinting and DNA methylation in Arabidopsis.” Plant J 71(5): 776-86.
Reinders, J., B. B. Wulff, et al., (2009). “Compromised stability of DNA methylation and transposon immobilization in mosaic Arabidopsis epigenomes.” Genes Dev 23(8): 939-50.
Richards, E. J. (2006). “Inherited epigenetic variation—revisiting soft inheritance.” Nat Rev Genet 7(5): 395-401.
Rodrigues, J. A., R. Ruan, et al., (2013). “Imprinted expression of genes and small RNA is associated with localized hypomethylation of the maternal genome in rice endosperm.” Proc Natl Acad Sci USA 110(19): 7934-9.
Ron, M., M. Alandete Saez, et al., (2010). “Proper regulation of a sperm-specific cis-nat-siRNA is essential for double fertilization in Arabidopsis.” Genes Dev 24(10): 1010-21.
Satyaki, P. R. V. and M. Gehring (2019). “Paternally Acting Canonical RNA-Directed DNA Methylation Pathway Genes Sensitize Arabidopsis Endosperm to Paternal Genome Dosage.” Plant Cell 31(7): 1563-1578.
Scott, R. J., M. Spielman, et al., (1998). “Parent-of-origin effects on seed development in Arabidopsis thaliana.” Development 125: 3329-3341.
Singh, M., S. Goel, et al., (2011). “Production of viable gametes without meiosis in maize deficient for an ARGONAUTE protein.” Plant Cell 23(2): 443-58.
Song, X., P. Li, et al., (2012). “Roles of DCL4 and DCL3b in rice phased small RNA biogenesis.”
Plant J 69(3): 462-74.
Steward, N., T. Kusano, et al., (2000). “Expression of ZmMET1, a gene encoding a DNA methyltransferase from maize, is associated not only with DNA replication in actively proliferating cells, but also with altered DNA methylation status in cold-stressed quiescent cells.” Nucleic Acids Res 28(17): 3250-9.
Stroud, H., M. V. Greenberg, et al., (2013). “Comprehensive analysis of silencing mutants reveals complex regulation of the Arabidopsis methylome.” Cell 152(1-2): 352-64.
Suter, L. and A. Widmer (2013). “Environmental heat and salt stress induce transgenerational phenotypic changes in Arabidopsis thaliana.” PLoS One 8(4): e60364.
Tao, Z., L. Shen, et al., (2017). “Embryonic epigenetic reprogramming by a pioneer transcription factor in plants.” Nature 551(7678): 124-128.
Teerawanichpan, P., P. Krittanai, et al., (2009). “Purification and characterization of rice DNA methyltransferase.” Plant Physiol Biochem 47(8): 671-80.
Ten Hove, C. A., K. J. Lu, et al., (2015). “Building a plant: cell fate specification in the early Arabidopsis embryo.” Development 142(3): 420-30.
Van Ex, F., Y. Jacob, et al., (2011). “Multiple roles for small RNAs during plant reproduction.” Curr Opin Plant Biol 14(5): 588-93.
Verhoeven, K. J., J. J. Jansen, et al., (2010). “Stress-induced DNA methylation changes and their heritability in asexual dandelions.” New Phytol 185(4): 1108-18.
Vongs, A., T. Kakutani, et al., (1993). “Arabidopsis thaliana DNA methylation mutants.” Science 260: 1926-1928.
Vu, T. M., M. Nakamura, et al., (2013). “RNA-directed DNA methylation regulates parental genomic imprinting at several loci in Arabidopsis.” Development 140(14): 2953-60.
Walker, J., H. Gao, et al., (2018). “Sexual-lineage-specific DNA methylation regulates meiosis in Arabidopsis.” Nat Genet 50(1): 130-137.
Williams, B. P., D. Pignatta, et al., (2015). “Methylation-sensitive expression of a DNA demethylase gene serves as an epigenetic rheostat.” PLoS Genet 11(3): e1005142.
Xiao, W., R. C. Brown, et al., (2006). “Regulation of seed size by hypomethylation of maternal and paternal genomes.” Plant Physiol 142(3): 1160-8.
Xiao, W., K. D. Custard, et al., (2006). “DNA methylation is critical for Arabidopsis embryogenesis and seed viability.” Plant Cell 18(4): 805-14.
Xiao, W. Y., M. Gehring, et al., (2003). “Imprinting of the MEA polycomb gene is controlled by antagonism between MET1 methyltransferase and DME glycosylase.” Developmental Cell 5(6): 891-901.
Xie, Z., E. Allen, et al., (2005). “DICER-LIKE 4 functions in trans-acting small interfering RNA biogenesis and vegetative phase change in Arabidopsis thaliana.” Proc Natl Acad Sci USA 102(36): 12984-9.
Xin, M., R. Yang, et al., (2014). “Dynamic parent-of-origin effects on small interfering RNA expression in the developing maize endosperm.” BMC Plant Biol 14: 192.
Zemach, A., M. Y. Kim, et al., (2013). “The Arabidopsis nucleosome remodeler DDM1 allows DNA methyltransferases to access H1-containing heterochromatin.” Cell 153(1): 193-205.
Zemach, A., M. Y. Kim, et al., (2010). “Local DNA hypomethylation activates genes in rice endosperm.” Proc Natl Acad Sci USA 107(43): 18729-34.
Zhai, J., H. Zhang, et al., (2015). “Spatiotemporally dynamic, cell-type-dependent premeiotic and meiotic phasiRNAs in maize anthers.” Proc Natl Acad Sci USA 112(10): 3146-51.
Zhang, M., S. Xie, et al., (2014). “Genome-wide high resolution parental-specific DNA and histone methylation maps uncover patterns of imprinting regulation in maize.” Genome Res 24(1): 167-76.
Zhang, X. and S. E. Jacobsen (2006). “Genetic analyses of DNA methyltransferases in Arabidopsis thaliana.” Cold Spring Harb Symp Quant Biol 71: 439-47.
Zhou, M., A. M. S. Palanca, et al., (2018). “Locus-specific control of the de novo DNA methylation pathway in Arabidopsis by the CLASSY family.” Nat Genet 50(6): 865-873.
Zilberman, D. (2004). “Role of Arabidopsis ARGONAUTE4 in RNA-directed DNA methylation triggered by inverted repeats.” Curr. Biol. 14: 1214-1220.
Drews, G. N., D. Wang, et al., (2011). “Identification of genes expressed in the angiosperm female gametophyte.” J Exp Bot 62(5): 1593-9.
Drews, G. N. and R. Yadegari (2002). “Development and function of the Angiosperm female gametophyte.” Annu. Rev. of Genetics 36: 99-124.
Gehring, M., Y. Choi, et al., (2004). “Imprinting and Seed Development.” Plant Cell 16: 10.1105/tpc.017988.
Jofuku, K. D., P. K. Omidyar, et al., (2005). “Control of seed mass and seed yield by the floral homeotic gene APETALA2.” Proc Natl Acad Sci USA 102(8): 3117-22.
Mizukami, Y. and R. L. Fischer (2000). “Plant organ size control: AINTEGUMENTA regulates growth and cell numbers during organogenesis.” Proc. Natl. Acad. Sci. USA 97: 942-947.
Ohto, M. A., R. L. Fischer, et al., (2005). “Control of seed mass by APETALA2.” Proc Natl Acad Sci USA 102(8): 3123-8.
Schruff, M. C., M. Spielman, et al., (2006). “The AUXIN RESPONSE FACTOR 2 gene of Arabidopsis links auxin signalling, cell division, and the size of seeds and other organs.” Development 133(2): 251-61.
Finnegan, E. J. and E. S. Dennis (1993). “Isolation and identification by sequence homology of a putative cytosine methyltransferase from Arabidopsis thaliana.” Nucl. Acids Res. 21: 2383-2388.
Finnegan, E. J. and K. A. Kovac (2000). “Plant DNA Methyltransferases.” Plant Mol Biol 43: 189-201.
Gehring, M., J. H. Huh, et al., (2006). “DEMETER DNA glycosylase establishes MEDEA polycomb gene self-imprinting by allele-specific demethylation.” Cell 124(3): 495-506.
Haig, D. and M. Westoby (1991). “Genomic imprinting in endosperm: its effect on seed development in crosses between species, and between different ploidies of the same species, and its implications for the evolution of apomixis.” Phil. Trans. R. Soc. Lond. B 333: 1-14.
Huang, Y., L. Ji, et al., (2009). “Structural insights into mechanisms of the small RNA methyltransferase HEN1.” Nature 461(7265): 823-7.
Ji, L. and X. Chen (2012). “Regulation of small RNA stability: methylation and beyond.” Cell Res 22(4): 624-36.
Jullien, P. E. and F. Berger (2006). “Parental genome dosage imbalance deregulates imprinting in Arabidopsis.” PLoS Genet 6(3): e1000885.
Kohler, C., D. R. Page, et al., (2005). “The Arabidopsis thaliana MEDEA Polycomb group protein controls expression of PHERES1 by parental imprinting.” Nat Genet 37(1): 28-30.
Li, E. (2002). “Chromatin Modification and Epigenetic Reprogramming in Mammalian Development.” Nat. Rev. Genet. 3: 662-673.
Martienssen, R. A. and V. Colot (2001). “DNA methylation and epigenetic inheritance in plants and filamentous fungi.” Science 293: 1070-1074.
Reik, W., W. Dean, et al., (2001). “Epigenetic reprogramming in mammalian development.” Science 293: 1089-1093.
Zemach, A., M. Y. Kim, et al., (2010). “Local DNA hypomethylation activates genes in rice endosperm.” Proc Natl Acad Sci USA 107(43): 18729-34.
Zemach, A., I. E. McDaniel, et al., (2010). “Genome-wide evolutionary analysis of eukaryotic DNA methylation.” Science 328(5980): 916-9.

1. A modified plant cell comprising gene with a mutation as compared to a comparable plant cell that results in the increase or decrease in the expression and/or activity of a corresponding gene product as compared to said comparable plant cell selected from the group consisting of ARF8, YDK1, RNA Pol IV, RNA Pol V, DCL3, AGO6, CMT3 and DRM2.

2. The modified plant cell of claim 1, wherein the mutation results in an increase in expression or activity of said corresponding gene product.

3. The modified plant cell of claim 1, wherein the mutation results in a decrease in expression or activity of said corresponding gene product.

4. The modified plant cell of claim 1, wherein the plant is Arabidopsis thaliana.

5. The modified plant cell of claim 1, the plant is corn, soybean, canola, rice, wheat, oat or barley.

6. The modified plant cell of claim 1, wherein the plant cell is homozygous for said mutation.

7. The modified plant cell of claim 1, wherein the plant cell is heterozygous for said mutation.

8. The modified plant cell of claim 1, wherein more than mutation is present in said plant cell.

9. The modified plant cell of claim 1, wherein more than one corresponding gene product has modified expression or activity in said plant cell.

10. The modified plant cell of claim 1, wherein said mutation is:
(a) located in said gene result in an amino acid substitution or truncation of said corresponding gene product;
(b) located in a regulatory sequence that controls expression of said corresponding gene product; or
(c) a heterologous nucleic acid encoding an agonist or antagonist of expression and/or activity of said corresponding gene product.

11. A living plant comprising the modified plant cell of claim 1.

12. A seed obtained from propagating a plant according to claim 11.

13. A plant obtained by breeding two plants, at least one of said two plants is a plant according to claim 11.

14. A plant obtained by breeding two plants, wherein both of said two plants are plants according to claim 11.

15. Progeny, a descendant, part or propagule of claim 11.

16. A method of propagating a plant comprising:
(a) planting a seed comprising a gene with mutation as compared to a comparable plant cell that results in the increase or decrease in the expression or activity of a corresponding gene product as compared to said comparable plant cell, wherein the gene is selected from the group consisting of ARF8, YDK1, RNA Pol IV, RNA Pol V, DCL3, AGO6, CMT3 and DRM2; and
(b) cultivating a plant grown from said seed.

17. A method of increasing or decreasing in the expression and/or activity of a gene product in a plant cell selected from the group consisting of ARF8, YDK1, RNA Pol IV, RNA Pol V, DCL3, AGO6, CMT3 and DRM2 comprising contacting said plant cell with an agonist or antagonist of expression and/or activity of said gene product.

18. The method of claim 17, wherein said plant cell is located in a living, intact plant.

19. The method of claim 17, wherein said agonist or antagonist is an organochemcial compound.

20. The method of claim 17, wherein said agonist or antagonist is a nucleic acid, such as an mRNA or an expression vector.

21. The method of claim 17, wherein said agonist or antagonist is a protein or peptide.
